SEQUENCE LISTING (1 sequence)

SEQ ID NO: 1 - protein, 8 aa, Artificial Sequence
  Synthetic peptide
SIINFEKL

(19) United States
(12) Patent Application Publication
(10) Pub. No.: US 2011/0104293 A1
(43) Pub. Date: May 5, 2011

(54) SYNERGISTIC INDUCTION OF HUMORAL AND CELLULAR IMMUNITY BY COMBINATORIAL ACTIVATION OF TOLL-LIKE RECEPTORS

(76) Inventors: Bali Pulendran, Alpharetta, GA (US); Sudhir Kasturi, Atlanta, GA (US); Niren Murthy, Atlanta, GA (US)

(21) Appl. No.: 13/002,211

(22) PCT Filed: Jul. 1, 2009
(86) PCT No.: PCT/US09/49431

§ 371 Date: Dec. 30, 2010

Related U.S. Application Data
(60) Provisional application No. 61/077,411, filed on Jul. 1, 2008.

(51) Int. Cl. A61K 39/00; A61K 9/50; A61K 39/145; A61K 39/07; A61P 35/00; A61P 31/16; A61P 31/04

(52) U.S. Cl. 424/490; 424/184.1; 424/497; 424/277.1; 424/209.1; 424/210.1; 424/246.1

(57) ABSTRACT

Described herein are compositions that include a selected antigen, a TLR4 ligand and a TLR7/TLR8 ligand, wherein the antigen and TLR ligands are encapsulated in nanoparticles. Co-administration of both a TLR4 ligand and a TLR7/TLR8 ligand results in the synergistic induction of humor and cellular immunity as evidenced by an increase in pro-inflammatory cytokine production, an increase in the number of CD8⁺ T effector and T memory cells, an increase in titer of antigen-specific antibodies, an increase in antibody affinity, an increase in the proliferation of naïve B cells and/or a significant enhancement in the persistence of antibody and T cell responses. The compositions and methods provided herein can be used to stimulate an immune response such as an immune response to a pathogen or a tumor.

### CROSS REFERENCE TO RELATED APPLICATION

This application claims the benefit of U.S. Provisional Application No. 61/077,411, filed Jul. 1, 2008, which is herein incorporated by reference in its entirety.

### STATEMENT OF GOVERNMENT SUPPORT

This invention was made with government support under 3U54-AI-057157-06S1 awarded by the National Institutes of Health. The government has certain rights in the invention.

### FIELD

This disclosure concerns compositions comprising nanoparticles loaded with toll-like receptor ligands (TLR) and the synergistic effects of the TLR ligands in eliciting an immune response.

### BACKGROUND

The hallmark of many highly effective vaccines is the induction of robust cellular and humoral immune responses. Most successful empirically derived vaccines, such as the smallpox or yellow fever virus vaccines stimulate polyvalent immune responses, however achieving such a response with synthetic vaccines has been a challenge. The limitation for many synthetic vaccines is that most current adjuvants do not stimulate both cellular and humoral immunity. Aluminium hydroxide-based adjuvants such as alum, which for many decades have been the only licensed adjuvants for clinical use, do not stimulate strong cellular immune responses. Thus, a need remains for the development of novel adjuvants that stimulate robust humoral and cellular responses for the control of infectious diseases.

There is evidence that toll-like receptors (TLR5) play a pivotal role in shaping the host immune response to a pathogen or a vaccine (Beutler, Nature 430:257-263, 2004; Kaisho and Akira, J. Allergy Clin. Immunol. 117:979-987, 2006; Pulendran and Ahmed, Cell 124(4):849-63, 2006; Medzhitov, Nat. Rev. Immunol. 1:135-145, 2001). Much of the understanding of the mechanisms by which this occurs has arisen from experiments that probe the response of immune cells to a single TLR ligand. However, microbes and vaccines do not simply stimulate a single TLR, but rather stimulate combinations of different TLR5. Thus, provided herein are compositions comprising nanoparticles loaded with a combination of TLR ligands and their methods of use.

### SUMMARY

Provided herein are compositions for stimulating an immune response to an antigen. The compositions include the antigen, a TLR4 ligand, and a TLR7/TLR8 ligand. In some embodiments, the antigen, TLR4 ligand and TLR7/TLR8 ligand are encapsulated by nanoparticles. Also provided herein is a method of stimulating an immune response to an antigen in a subject, that can include administering to the subject a composition comprising the antigen, a TLR4 ligand, and a TLR7/TLR8 ligand, wherein the antigen, TLR4 ligand and TLR7/TLR8 ligand are encapsulated by nanoparticles. As described herein, administration of both a TLR4 ligand and a TLR7/TLR8 ligand results in a synergistic stimulation of an antigen-specific immune response as compared to administration of a single TLR ligand.

In some cases, the TLR4 ligand is encapsulated in the same nanoparticles as the TLR7/TLR8 ligand. In other cases, the TLR4 ligand is encapsulated in different nanoparticles as the TLR7/TLR8 ligand. In some embodiments, the antigen is encapsulated by the same nanoparticles as the TLR ligands. In other embodiments, the antigen is encapsulated by different nanoparticles as the TLR ligands. Exemplary nanoparticles are made of biocompatible and biodegradable polymeric materials. In some embodiments, the nanoparticles are polymeric nanoparticles, such as poly(lactic acid) or poly(glycolic acid) nanoparticles. In particular examples, the nanoparticles are poly(D,L-lactic-co-glycolic acid) (PLGA) nanoparticles. In some embodiments, the TLR4 ligand is MPL and the TLR7/TLR8 ligand is R837. The antigen encapsulated by the nanoparticles can be any type of antigen, including a tumor antigen or an antigen from a pathogen.

In some embodiments, stimulating an immune response is indicated by an increase in the production of pro-inflammatory cytokines; an increase in the number of CD8⁺ T effector cells; an increase in the number of CD8⁺ T memory cells; an increase in the number of CD4⁺ T memory cells; an increase in titer of antigen-specific antibodies; an increase in antigen-specific antibody affinity; an increase in titer of neutralizing antibodies; an increase in the proliferation of naïve B cells; an increase in persistence of antigen-specific B cells; an increase in the number of germinal centers; an increase in the number of antibody secreting cells; or a combination of two or more thereof. In some embodiments, the methods further include detecting an indicator of an immune response in a sample obtained from the subject.

The foregoing and other features will become more apparent from the following detailed description of several embodiments, which proceeds with reference to the accompanying figures.

### BRIEF DESCRIPTION OF THE FIGURES

FIG. 1 is a series of FACS plots showing that co-delivery of PLGA nanoparticle-encapsulated TLR ligands (MPL, a TLR4 ligand; or R837, a TLR7 ligand; or both) enhances delivery of PLGA-encapsulated antigen (Ova) to conventional DCs.

FIG. 2 is a series of FACS plots showing that co-delivery of PLGA nanoparticle-encapsulated TLR ligands (MPL, R837 or both) enhances delivery of PLGA-encapsulated antigen (Ova) to plasmacytoid DCs.

FIG. 3 is a series of FACS plots showing that co-delivery of PLGA nanoparticle-encapsulated TLR ligands (MPL, R837 or both) enhances delivery of PLGA-encapsulated antigen (Ova) to dermal, Langerhans, myeloid and lymphoid DCs.

FIGS. 4A-4D are graphs showing that delivery of PLGA nanoparticles containing both MPL and R837 with PLGA nanoparticles containing antigen (Ova) results in synergistic enhancement in the production of the pro-inflammatory cytokines IL-12p70 (A), IFN-α (B), IL-6 (C) and TNF-α (D) by CD1c⁺ DCs, relative to delivery of PLGA nanoparticles containing a single TLR ligand.

FIG. 5A is a FACS plot showing that treatment of CD11c⁺ DCs with PLGA nanoparticles containing both MPL and R837 results in synergistic production of IL-12, relative to treatment with PLGA nanoparticles containing a single TLR ligand. FIG. 5B is a graph quantifying the percentage of CD11c⁺ DCs positive for IL-12 expression under each condition.

FIGS. 6A and 6B are graphs showing that the combined delivery of TLR ligands MPL and R837 in PLGA nanoparticles results in the synergistic enhancement of IFN-γ production by memory CD8⁺ T cells (B), but not by primary CD8⁺ T cells (A), at a suboptimal antigen dose (10 μg). IFN-γ production by memory CD8⁺ T cells was significantly greater following treatment with PLGA nanoparticles containing both TLR ligands relative to treatment with PLGA nanoparticles containing a single TLR ligand, and to treatment with soluble TLR ligand(s).

FIG. 7 shows representative FACS plots of CD8⁺ T cells obtained from one mouse per treatment group for the experiment shown in FIG. 6.

FIGS. 8A-8C are graphs showing serum antibody isotype profiles of mice 28 days after immunization with PLGA-encapsulated ovalbumin (Ova) in combination with PLGA-encapsulated MPL, R837 or both MPL and R837. Control animals were treated with soluble Ova or PLGA-encapsulated Ova only. Shown are antibody titers of IgG_{2c} (A), IgG_{2b} (B) and IgG₁ (C). Delivery of nanoparticles containing both MPL and R837 resulted in synergistic enhancement of IgG_{2c}, IgG_{2b} and IgG₁ antibody titers relative to delivery of a single TLR ligand.

FIGS. 9A-9C are graphs showing serum antibody isotype profiles of mice 28 days after a boost immunization with PLGA-encapsulated Ova in combination with PLGA-encapsulated MPL, R837 or both MPL and R837. Shown are antibody titers of IgG_{2c} (A), IgG_{2b} (B) and IgG₁ (C). Delivery of nanoparticles containing both MPL and R837 resulted in synergistic enhancement of IgG_{2c, IgG}_{2b} and IgG₁ antibody titers relative to delivery of a single TLR ligand.

FIGS. 10A-10C are graphs showing serum antibody isotype profiles of mice 28 days after immunization with PLGA-encapsulated anthrax protective antigen (PA) in combination with PLGA-encapsulated MPL, R837 or both MPL and R837. Control animals were treated with soluble PA or PLGA-encapsulated PA only. Shown are antibody titers of IgG_{2b} (A), IgG₂ₐ (B) and IgG₁ (C). Delivery of nanoparticles containing both MPL and R837 resulted in synergistic enhancement of IgG_{2b}, IgG₂ₐ and IgG₁ antibody titers relative to delivery of a single TLR ligand.

FIGS. 11A-11C are graphs showing serum antibody isotype profiles of mice 28 days after a boost immunization with PLGA-encapsulated PA in combination with PLGA-encapsulated MPL, R837 or both MPL and R837. Shown are antibody titers of IgG_{2b} (A), IgG₂ₐ (B) and IgG₁ (C). Delivery of nanoparticles containing both MPL and R837 resulted in synergistic enhancement of IgG_{2b}, IgG₂ₐ and IgG₁ antibody titers relative to delivery of a single TLR ligand.

FIG. 12 is a graph illustrating binding affinity (including dissociation and association rates) of serum antibodies obtained from mice immunized with soluble or PLGA-encapsulated PA alone or in combination with PLGA-encapsulated MPL (TLR4), R837 (TLR7) or both MPL and R837. Treatment with nanoparticles containing both TLR ligands results in the production of high affinity antibodies relative to treatment with nanoparticles containing a single TLR ligand.

FIG. 13A is a series of graphs showing IgG₂ₐ, IgG_{2b} and IgG₁ antibody titers after immunization with 0.1, 1.0 or 10 μg of PLGA-encapsulated avian influenza hemagglutinin (HA) in combination with PLGA-encapsulated MPL, R837 or both MPL and R837. Control animals were treated with soluble HA or PLGA-encapsulated HA only. Shown are antibody titers 28 days after primary immunization. Delivery of nanoparticles containing both MPL and R837 resulted in synergistic enhancement of IgG₂ₐ, IgG_{2b} and IgG₁ antibody titers relative to delivery of a single TLR ligand.

FIG. 13B is a series of graphs showing IgG₂ₐ, IgG_{2b} and IgG₁ antibody titers after immunization with 0.1, 1.0 or 10 μg of PLGA-encapsulated avian influenza hemagglutinin (HA) in combination with PLGA-encapsulated MPL, R837 or both MPL and R837. Control animals were treated with soluble HA or PLGA-encapsulated HA only. Shown are antibody titers 28 days after a boost immunization. Delivery of nanoparticles containing both MPL and R837 resulted in synergistic enhancement of IgG₂ₐ, IgG_{2b} and IgG₁ antibody titers relative to delivery of a single TLR ligand.

FIG. 13C is a graph illustrating binding affinity (including dissociation and association rates) of serum antibodies obtained from mice immunized with soluble or PLGA-encapsulated HA alone or in combination with PLGA-encapsulated MPL (TLR4), R837 (TLR7) or both MPL and R837. Treatment with nanoparticles containing both TLR ligands results in the production of high affinity antibodies relative to treatment with nanoparticles containing a single TLR ligand.

FIGS. 14A-14C are graphs showing polyclonal stimulation of purified splenic B cells following in vitro treatment with blank nanoparticles or nanoparticles containing MPL, R837 or both MPL and R837. Shown is proliferation (measured by CPM of incorporated ³H-thymidine) of wild-type (A), MyD88 knockout (B) and TRIF knockout (C) naïve B cells. Proliferation of MyD88 knockout B cells was significantly inhibited, while proliferation of TRIF knockout B cells was partially inhibited, relative to wild-type B cells.

FIGS. 15A-15C are graphs showing serum antibody isotype profiles of untreated mice and mice treated with soluble ovalbumin (Alum(Ova)), and wild-type, MyD88-deficient (MyD88KO) and TRIF-deficient (TRIFKO) mice treated with PLGA-encapsulated Ova in combination with PLGA-encapsulated MPL and R837. Shown are antibody titers of IgG₂ (A), IgG_{2b} (B) and IgG₁ (C). Antibody titers were significantly inhibited in MyD88-deficient and TRIF-deficient mice.

FIG. 16 is a graph showing the percentage of IFN-γ-positive CD8⁺ T cells following treatment with PLGA-encapsulated nanoparticles containing 10, 50 or 100 μg of Ova in combination with PLGA nanoparticles containing MPL, R837 or both.

FIG. 17 is a series of FACS plots showing that delivery of PLGA nanoparticles containing both MPL and R837 with PLGA nanoparticles containing antigen (Ova) results in a synergistic increase in the percentage of IFN-γ, TNF-α and IL-2 producing CD8⁺ T cells, relative to treatment with PLGA nanoparticles containing a single TLR ligand.

FIG. 18 is a series of FACS plots (A) and a graph (B) showing that delivery of PGLA nanoparticles containing both TLR ligands MPL and R837 synergistically enhances memory CD4+ T cell responses in vivo. Shown are the percentage of CD4⁺IFN-γ⁺ cells obtained from mice 8 weeks after boost immunization with PLGA nanoparticles containing Ova and PLGA nanoparticles containing MPL, R837, or both.

FIGS. 19A-19C are graphs showing IgG₂ (A), IgG_{2b} (B) and IgG₁ (C) antibody titers after immunization with 10 μg of PLGA-encapsulated Ova in combination with PLGA-encapsulated MPL, R837 or both MPL and R837, in wild-type (C57BL6) mice or CD11c-DTR mice. This demonstrates that CD11c⁺ DCs are required for TLR-mediated induction of antibody responses.

FIGS. 20A-20C are graphs showing IgG₂, (A), IgG_{2b} (B) and IgG₁ (C) antibody titers after immunization with 10 μg of PLGA-encapsulated Ova in combination with PLGA-encapsulated MPL, R837 or both MPL and R837, in wild-type (C57BL6) mice or Langerin-DTR mice. This demonstrates that Langerin⁺ DCs are required for TLR-mediated induction of antibody responses.

FIGS. 21A-21D are graphs showing antibody titers after immunization of C57BL6, IL-6^{-/-}, B6129 and IFNα/R^{-/-} mice with 10 μg of Ova encapsulated in PLGA nanoparticles in combination with PLGA-encapsulated MPL, R837 or both MPL and R837. This demonstrates that IL-6 and IFN-α are required for TLR-mediated induction of antibody responses.

FIGS. 22A-22C are graphs showing IgG₂, IgG_{2b} and IgG₁ antibody titers after immunization with 10 μg of PLGA-encapsulated Ova in combination with PLGA-encapsulated MPL, R837 or both MPL and R837 in CD4⁺ T cell-sufficient and CD4⁺ T cell-deficient mice. This demonstrates that CD4⁺ T-helper cells are required for TLR-mediated induction of antibody responses.

FIG. 23 is two graphs showing total IgG antibody titers following prime and boost immunization of mice transplanted with wild-type B cells, MyD88KO B cells or TRIFKO B cells. This demonstrates that both the MyD88 and TRIF mediated pathway of TLR signaling are required for TLR-mediated induction of antibody responses.

FIG. 24 is two graphs showing total IgG antibody titers following prime and boost immunization of mice transplanted with wild-type B cells, TLR4KO B cells, TLR7KO B cells or both TLR4KO B cells and TLR7KO B cells.

FIG. 25 is a series of FACS plots showing antigen-specific B cells responses following immunization with nanoparticle-encapsulated Ova and nanoparticle-encapsulated MPL+R837.

FIG. 26 is a series of FACS plots showing the percentage of ovalbumin-specific CD19⁺ B cells at 14 days post primary immunization (top row) or 8 weeks post secondary immunization (bottom row) with PLGA-encapsulated Ova in combination with PLGA-encapsulated MPL, R837 or both MPL and R837.

FIG. 27 is two graphs showing the number of germinal centers per lymph node at days 14 (D14) and 28 (D28) post-immunization with PLGA-encapsulated Ova in combination with PLGA-encapsulated MPL, R837 or both MPL and R837.

FIG. 28 is two graphs showing the number of antibody forming plasma cells at day 28 post primary immunization or 14 days post boost immunization with PLGA-encapsulated Ova in combination with PLGA-encapsulated MPL, R837 or both MPL and R837.

FIG. 29 is a graph showing the kinetics of formation of antibody forming plasma cells in mice immunized with PLGA-encapsulated Ova in combination with PLGA-encapsulated MPL, R837 or both MPL and R837.

FIG. 30 is two graphs showing persistence of antibody secreting cells in draining lymph nodes up to 1.5 years following immunization with PLGA-encapsulated Ova in combination with PLGA-encapsulated MPL+R837.

FIG. 31A is a graph showing virus neutralization titers in mice following immunization with 10 μg of PLGA-encapsulated HA in combination with PLGA-encapsulated MPL, R837 or both MPL and R837.

FIG. 31B is a graph showing virus neutralization titers in mice following immunization with 0.1, 1.0 or 10 μg of PLGA-encapsulated HA in combination with PLGA-encapsulated MPL and R837.

FIG. 32 is a graph showing that delivery of PLGA nanoparticles containing both MPL and R848 (a ligand that stimulates both TLR7 and TLR8) with PLGA nanoparticles results in synergistic enhancement in the production of the pro-inflammatory cytokine IL-12p70 in human monocyte derived DCs.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the amino acid sequence of an ovalbumin-specific class I peptide.

### DETAILED DESCRIPTION

### I. Abbreviations

| | |
|---|---|
| AFP | Alphafetoprotein |
| APC | Antigen presenting cell |
| ASC | Antigen secreting cell |
| BCA | Bicinchoninic acid |
| CEA | Carcinoembryonic antigen |
| CPM | Counts per minute |
| DC | Dendritic cell |
| DMSO | Dimethyl sulfoxide |
| DT | Diphtheria toxin |
| DTR | Diphtheria toxin receptor |
| ELISA | Enzyme-linked immunosorbent assay |
| ETA | Epithelial tumor antigen |
| FACS | Fluorescence-activated cell sorting |
| HA | Hemagglutinin |
| HAI | Hemagglutinin inhibition |
| HCV | Hepatitis C virus |
| HIV | Human immunodeficiency virus |
| HSV | Herpes simplex virus |
| IGF | Insulin growth factor |
| KO | Knockout |
| LPS | Lipopolysaccharide |
| MAGE | Melanoma-associated antigen |
| MPL | Monophosphoryl lipid A |
| OVA | Ovalbumin |
| PBC | Peripheral blood cell |
| PBS | Phosphate-buffered saline |
| PBMC | Peripheral blood mononuclear cells |
| PCTA-1 | Prostate carcinoma tumor antigen-1 |
| PDCA | Plasmacytoid dendritic cell antigen |
| PGA | Polyglycolide |
| PLA | Poly(lactic acid) |
| PLGA | Poly(D,L-lactic-co-glycolic acid) |
| PRAME | Preferentially expressed antigen of melanoma |
| PSA | Prostate-specific antigen |
| PVA | Poly(vinyl alcohol) |
| SARS | Severe acute respiratory syndrome |
| SDS | Sodium dodecyl sulfate |
| TLR | Toll-like receptor |
| TRIF | TIR-domain-containing adapter-inducing interferon-β |
| WT1 | Wilms tumor 1 |

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN1-56081-569-8). In order to facilitate review of the various embodiments of the disclosure, the following explanations of specific terms are provided:

Administration: The introduction of a composition into a subject by a chosen route. For example, if the chosen route is intravenous, the composition is administered by introducing the composition into a vein of the subject.

Animal: Living multi-cellular vertebrate organisms, a category that includes, for example, mammals and birds. The term mammal includes both human and non-human mammals. Similarly, the term “subject” includes both human and veterinary subjects.

Antibody: A polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

Antibody secreting cell (ASC): Refers to any type of cell that is capable of producing and secreting antibodies. ASCs can be found, for example, in the lymph nodes.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens.

Binding affinity: Affinity of an antibody for an antigen. In one embodiment, affinity is calculated by a modification of the Scatchard method described by Frankel et al. (Mol. Immunol., 16:101-106, 1979). In another embodiment, binding affinity is measured by an antigen/antibody dissociation rate. In another embodiment, a high binding affinity is measured by a competition radioimmunoassay. In another embodiment, binding affinity is measured by ELISA.

Cancer or tumor antigen: A cancer or tumor antigen is an antigen that can stimulate tumor-specific T-cell immune responses. Exemplary tumor antigens include, but are not limited to, RAGE-1, tyrosinase, MAGE-1, MAGE-2, NY-ESO-1, Melan-A/MART-1, glycoprotein (gp) 75, gp100, beta-catenin, PRAME, MUM-1, WT-1, CEA, and PR-1. Additional tumor antigens are known in the art (for example see Novellino et al., Cancer Immunol. Immunother. 54(3):187-207, 2005) and are described below. As used herein, tumor antigens include those not yet identified. Cancer antigen and tumor antigen are used interchangeably herein.

CD8⁺ T effector cells: Activated T cells that express CD8. During an immune response, effector T cells divide rapidly and secrete cytokines to modulate the immune response. T effector cells are also known as T helper cells.

CD8⁺ or CD4⁺ T memory cells: Antigen-specific T cells that persist long-term after an immune response. Upon re-exposure to the antigen, memory T cells expand and become T effector cells.

Cytokines: Proteins produced by a wide variety of hematopoietic and non-hematopoietic cells that affect the behavior of other cells. Cytokines are important for both the innate and adaptive immune responses.

Delivered simultaneously: As used herein, simultaneous delivery of two or more compounds or compositions refers to delivery of the compounds or compositions at the same time, or in immediate succession, such as within 1 minute, or 5 minutes, or 15 minutes of each other.

Detecting an increase: As used herein, “detecting an increase” in an indicator of an immune response refers to detecting an increase in the indicator (such as cytokines, antibodies or a particular cell type) in a sample obtained from a subject relative to a control. The control can be a sample obtained from the subject prior to immunization, a control sample obtained from a non-immunized subject or a standard value.

Encapsulated: As used herein, a molecule “encapsulated” in a nanoparticle refers to a molecule (such as an antigen or a TLR ligand) that is either contained within the nanoparticle or attached to the surface of the nanoparticle, or a combination thereof.

Germinal center: The area in the center of a lymph node containing aggregations of actively proliferating lymphocytes. Germinal centers are the sites of antibody production and are populated mostly by B cells, but include a few T cells and macrophages.

Imiquimod (R837): A low molecular synthetic molecule that binds toll-like receptor (TLR) 7 and TLR8. R837 is an imidazoquinoline amine analogue to guanosine. The chemical name of R837 is 1-isobutyl-1H-imidazo[4,5-c]quinolin-4-amine. R837 is commercially available, such as by InvivoGen, San Diego, Calif.

Immune response: A response of a cell of the immune system, such as a B cell or T cell, to a stimulus. In some embodiments, the response is specific for a particular antigen (an “antigen-specific response”). In some embodiments, an immune response is a T cell response, such as a CD8+ response. In another embodiment, the response is a B cell response, and results in the production of antigen-specific antibodies. As used herein, “stimulating an immune response” refers to promoting or enhancing the response of the cells of the immune system to a stimulus, such as an antigen. Stimulation of the immune response can be indicated by, for example, an increase in the production of pro-inflammatory cytokines; an increase in the number of CD8⁺ T effector cells; an increase in the number of CD8⁺ T memory cells; an increase in the number of CD4⁺ T memory cells; an increase in titer of antigen-specific antibodies; an increase in antigen-specific antibody affinity; an increase in titer of neutralizing antibodies; an increase in the proliferation of naïve B cells; an increase in persistence of antigen-specific B cells; an increase in the number of germinal centers; an increase in the number of antibody secreting cells; or a combination thereof. The increase in the indicator of an immune response is relative to a control, such as a value observed before administration of the antigen or in the absence of treatment. As used herein, “an indicator of an immune response” refers to a measurable effect of an immune response, such as cytokine production, proliferation of T cells or B cells, activation of T cells, antibody production, increased antibody affinity, or a combination thereof.

Immunogen: A compound, composition, or substance which is capable, under appropriate conditions, of stimulating an immune response, such as the production of antibodies or a T-cell response in an animal, including compositions that are injected or absorbed into an animal, or otherwise administered to an animal.

Isolated: An “isolated” biological component, such as a nucleic acid, protein (including antibodies) or organelle that has been substantially separated or purified away from other biological components in the environment (such as a cell) in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Monophosphoryl lipid A (MPL): A low-toxicity derivative of lipid A, a component of LPS. MPL is a phosphorus-containing polyheterocyclic compound having pendant long chain, aliphatic ester and amide groups, and is obtained as an endotoxic extract from enterobacteria. MPL can be prepared as described in U.S. Pat. Nos. 4,436,727 and 4,436,728, or is commercially available (Avanti Lipids, Alabaster, Ala.).

Nanoparticle: A particle less than about 1000 nanometers (nm) in diameter. Exemplary nanoparticles for use with the methods provided herein are made of biocompatible and biodegradable polymeric materials. In some embodiments, the nanoparticles are PLGA nanoparticles. As used herein, a “polymeric nanoparticle” is a nanoparticle made up of repeating subunits of a particular substance or substances. “Poly(lactic acid) nanoparticles” are nanoparticles having repeated lactic acid subunits. Similarly, “poly(glycolic acid) nanoparticles” are nanoparticles having repeated glycolic acid subunits.

Neoplasia, malignancy, cancer or tumor: The result of abnormal and uncontrolled growth of cells. Neoplasia, malignancy, cancer and tumor are often used interchangeably and refer to abnormal growth of a tissue or cells that results from excessive cell division. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, menangioma, neuroblastoma and retinoblastoma).

Neutralizing antibody: A type of antibody that is capable of inhibiting or preventing infectivity of a microorganism, such as a virus. In some cases, a neutralizing antibody prevents a virus from penetrating a cell.

Pathogen: A biological agent that causes disease or illness to its host. Pathogens include, for example, bacteria, viruses, fungi, protozoa and parasites. Pathogens are also referred to as infectious agents.

Examples of pathogenic viruses include, but are not limited to those in the following virus families: Retroviridae (for example, human immunodeficiency virus (HIV), human T-cell leukemia viruses; Picornaviridae (for example, polio virus, hepatitis A virus, hepatitis C virus, enteroviruses, human coxsackie viruses, rhinoviruses, echoviruses, foot-and-mouth disease virus); Caliciviridae (such as strains that cause gastroenteritis, including Norwalk virus); Togaviridae (for example, alphaviruses (including chikungunya virus, equine encephalitis viruses, Simliki Forest virus, Sindbis virus, Ross River virus), rubella viruses); Flaviridae (for example, dengue viruses, yellow fever viruses, West Nile virus, St. Louis encephalitis virus, Japanese encephalitis virus, Powassan virus and other encephalitis viruses); Coronaviridae (for example, coronaviruses, severe acute respiratory syndrome (SARS) virus; Rhabdoviridae (for example, vesicular stomatitis viruses, rabies viruses); Filoviridae (for example, Ebola virus, Marburg virus); Paramyxoviridae (for example, parainfluenza viruses, mumps virus, measles virus, respiratory syncytial virus); Orthomyxoviridae (for example, influenza viruses); Bunyaviridae (for example, Hantaan viruses, Sin Nombre virus, Rift Valley fever virus, bunya viruses, phleboviruses and Nairo viruses); Arenaviridae (such as Lassa fever virus and other hemorrhagic fever viruses, Machupo virus, Junin virus); Reoviridae (e.g., reoviruses, orbiviurses, rotaviruses); Birnaviridae; Hepadnaviridae (hepatitis B virus); Parvoviridae (parvoviruses); Papovaviridae (papilloma viruses, polyoma viruses, BK-virus); Adenoviridae (adenoviruses); Herpesviridae (herpes simplex virus (HSV)-1 and HSV-2; cytomegalovirus; Epstein-Barr virus; varicella zoster virus; and other herpes viruses, including HSV-6); Poxyiridae (variola viruses, vaccinia viruses, pox viruses); and Iridoviridae (such as African swine fever virus); Astroviridae; and unclassified viruses (for example, the etiological agents of spongiform encephalopathies, the agent of delta hepatitis (thought to be a defective satellite of hepatitis B virus).

Examples of bacterial pathogens include, but are not limited to: Helicobacter pylori, Escherichia coli, Vibrio cholerae, Borelia burgdorferi, Legionella pneumophilia, Mycobacteria sps (such as. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Bordetella pertussis, Shigella flexnerii, Shigella dysenteriae and Actinomyces israelli.

Examples of fungal pathogens include, but are not limited to: Cryptococcus neoformans, Histoplasma capsulatum, Coccidioides immitis, Blastomyces dermatitidis, Chlamydia trachomatis, Candida albicans.

Other pathogens (such as parasitic pathogens) include, but are not limited to: Plasmodium falciparum, Plasmodium vivax, Trypanosoma cruzi and Toxoplasma gondii.

Pharmaceutical agent: A chemical compound or composition capable of inducing a desired therapeutic or prophylactic effect when properly administered to a subject or a cell.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition, 1975, describes compositions and formulations suitable for pharmaceutical delivery of the nanoparticles disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Poly(D,L-lactic-co-glycolic acid) (PLGA): A biodegradable polymer approved for human use as a suture material and as a controlled-release drug delivery system. Microparticles and nanoparticles made of PLGA are efficiently phagocytosed by antigen presenting cells (APCs), such as dendritic cells (DCs). PLGA nanoparticles are suitable for delivery of a variety of biological molecules, including, but not limited to recombinant proteins, peptides, and plasmid DNA.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Pro-inflammatory cytokines: Cytokines produced predominantly by activated immune cells that are involved in the amplification of inflammatory reactions. Pro-inflammatory cytokines include, but are not limited to IL-1, IL-6, IL-8, IL-12, IFN-α, TNF-α, and TGF-β.

Purified: The term purified does not require absolute purity; rather, it is intended as a relative term. Thus, for example, a purified peptide preparation is one in which the peptide or protein is more enriched than the peptide or protein is in its natural environment within a cell. In some embodiments, a preparation is purified such that the protein or peptide represents at least 50%, at least about 75%, at least about 90%, at least about 95% or at least about 99% of the total peptide or protein content of the preparation.

Sample: As used herein, a “sample” obtained from a subject refers to a cell, fluid or tissue sample. Bodily fluids include, but are not limited to, blood, serum, urine and saliva.

Subject: Living multi-cellular vertebrate organisms, a category that includes both human and veterinary subjects, including human and non-human mammals.

Synergistic stimulation: As used herein “synergistic stimulation” of an immune response as a result of administration of two agents (such as a TLR4 ligand and a TLR8 ligand) in the presence of an antigen refers to an increase in the immune response that is greater than the sum increase that would occur upon administration of the agents individually in the presence of the antigen.

Therapeutically effective amount: A quantity of a specific substance sufficient to achieve a desired effect in a subject being treated. For instance, this can be the amount necessary to elicit an effective immune response against an antigen.

Toll-like receptors (TLR5): TLR5 are a class of single membrane-spanning non-catalytic receptors that recognize structurally conserved molecules derived from microbes and which activate immune responses. TLR5 play an important role in the innate immune system. Ligands for TLR5 include both natural (e.g., LPS, double-stranded RNA) and synthetic (e.g., poly(I:C), imidazoquinolines) ligands. For example, TLR4 ligands include LPS and lipid A. TLR7/TLR8 ligands include GU-rich single-stranded RNA, and imidazoquinolines (such as imiquimod (R837) and resiquimod (R848)). As used herein, “TLR7/TLR8 ligand” refers to a ligand that binds TLR7, TLR8 or both TLR7 and TLR8.

Vaccine: A preparation of immunogenic material capable of stimulating an immune response, administered for the prevention, amelioration, or treatment of infectious or other types of disease, such as cancer. The immunogenic material may include attenuated or killed microorganisms (such as bacteria or viruses), or antigenic proteins, peptides or DNA derived from them. Vaccines may elicit both prophylactic (preventative) and therapeutic responses. Methods of administration vary according to the vaccine, but may include inoculation, ingestion, inhalation or other forms of administration.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, GenBank Accession Numbers and other references mentioned herein are incorporated by reference in their entirety. In case of conflict, the present specification, including explanations of terms, will control. In addition, the materials, methods, and examples are illustrative only and not intended to be limiting.

### II. Introduction

Toll-like receptors (TLR5) are known to play a pivotal role in shaping the host immune response to a pathogen or a vaccine (Beutler, Nature 430:257-263, 2004; Kaisho and Akira, J. Allergy Clin. Immunol. 117:979-987, 2006; Pulendran and Ahmed, Cell 124(4):849-63, 2006; Medzhitov, Nat. Rev. Immunol. 1:135-145, 2001). However, little is known about the innate immune mechanisms that affect critical variables of the B cell response, such as memory B cell generation, affinity maturation, and induction of neutralizing antibodies. Such understanding is important for the rational design of vaccines that stimulate optimally effective B cell responses against various pathogens.

It is described herein that TLR ligands administered with an antigen can elicit antigen-specific antibody responses. Administration of combinations of TLR ligands results in a synergistic induction of antigen-specific CD8⁺ T cell responses, synergistic induction of antigen-specific antibody responses, and synergistic induction of high affinity and high avidity antibodies. In particular embodiments, it is described herein that (i) delivery of a TLR ligand in biodegradable PLGA nanoparticles results in profoundly enhanced antigen-specific CD8⁺ T cell and B cell responses, relative to delivery of the TLR ligand in a non-encapsulated (soluble) form; (ii) administration of PLGA nanoparticles containing two different TLR ligands results in a synergistic stimulation of antigen-specific CD8⁺ T cell, CD4⁺ T cell and B cell responses, relative to injection of nanoparticles containing an individual ligand; (iii) administration of PLGA nanoparticles containing two different TLR ligands results in a synergistic induction of high avidity/high affinity antibodies, relative to injection of nanoparticles containing an individual TLR ligand; and (iv) administration of PLGA nanoparticles containing two different TLR ligands results in a synergistic stimulation of dendritic cell and innate immune responses in vivo, relative to administration of nanoparticles containing an individual TLR ligand; (v) the combination of TLR ligands MPL and R837 induces persistent germinal centers and long lived antibody secreting cells in the draining lymph nodes of mice; (vi) the antibodies produced in response to delivery of the combination of MPL and R837 are of high avidity, are virus neutralizing and are synergistically enhanced in comparison with single TLR ligand treatment; and (vii) the synergistic enhancement of humoral immunity with the combination of TLR ligands is dependent on the presence of MyD88 and TRIF adaptor proteins and on the presence of TLR5 and signaling proteins in B cells.

The ability to induce high titers of high affinity antibodies is critical for conferring protective immunity against almost all pathogens. Therefore, the present disclosure of specific combinations of TLR ligands that synergistically stimulate high affinity antibody responses, and in particular embodiments, the use of polymeric nanoparticles that contain specific combinations of two different TLR ligands, addresses a critical challenge in vaccine development.

### III. Overview of Several Embodiments

Described herein is the finding that administration of a selected antigen and a combination of TLR ligands, such as a TLR4 ligand and a TLR7/TLR8 ligand, results in the synergistic enhancement of an antigen-specific immune response. In particular examples, the antigen and TLR ligands are administered in nanoparticles. The antigen and TLR ligands also can be administered using any other suitable delivery vehicle, such as a liposome or microparticle. Although administration of a single TLR ligand (such as encapsulated in a nanoparticle) enhances the immune response relative to administration of soluble antigen, administration of at least two TLR ligands results in an unexpectedly superior synergistic response.

In some embodiments, the combination of TLR ligands includes a TLR4 ligand and a TLR7/TLR8 ligand. In other embodiments, the combination of TLR ligands includes a TLR3 ligand and a TLR7/TLR8 ligand. In other embodiments, the combination of TLR ligands includes a TLR4 ligand and a TLR9 ligand. In other embodiments, the combination of TLR ligands includes a TLR3 ligand and a TLR9 ligand. Although exemplary combinations of TLR ligands are described herein, any combination of TLR ligands that results in a synergistic enhancement of an immune response is contemplated herein.

In particular, specific combinations of TLR ligands resulted in a synergistic induction of antigen-specific T cell responses, antigen-specific antibody responses, and high avidity antibodies. As described in particular examples herein, delivery of a TLR7/TLR8 ligand or a TLR4 ligand in biodegradable polymeric nanoparticles, such as PLGA nanoparticles, results in enhanced antigen-specific CD8⁺ T, CD4⁺ T cell and B cell responses, relative to delivery of the TLR ligand in an unencapsulated (soluble) form. However, administration of a mixture of PLGA nanoparticles containing both a TLR7/TLR8 ligand and a TLR4 ligand results in a synergistic stimulation of antigen-specific CD8⁺ T cell, CD4⁺ T cell and B cell responses, synergistic production of high avidity/affinity antibodies and neutralizing antibodies, and a synergistic stimulation of dendritic cell and innate immune responses in vivo, relative to administration of either TLR ligand alone. The current disclosure is the first demonstration of synergistic activation of B cell responses; synergistic induction of high affinity/avidity antibody responses; synergistic induction of neutralizing antibody responses; synergistic induction of enhanced persistence of the antibody response; and synergistic induction of antigen-specific CD8⁺ T cell and CD4⁺ T cell responses in vivo using a combination of TLR ligands.

Provided herein are compositions for stimulating an immune response to an antigen. The compositions include the target antigen, a TLR4 ligand and a TLR7/TLR8 ligand, for example wherein the antigen, the TLR4 ligand and the TLR7/TLR8 ligand are encapsulated in nanoparticles. In some embodiments, the TLR4 ligand is encapsulated in the same nanoparticles as the TLR7/TLR8 ligand.

In other embodiments, the TLR4 ligand is encapsulated in different nanoparticles as the TLR7/TLR8 ligand. In some embodiments, the antigen is encapsulated by the same nanoparticles as the TLR ligands. In other embodiments, the antigen is encapsulated by different nanoparticles as the TLR ligands. Exemplary nanoparticles are made of biocompatible and biodegradable polymeric materials. In some embodiments, the nanoparticles are poly(lactic acid) nanoparticles, poly(glycolic acid) nanoparticles, or both. In particular examples, the nanoparticles are poly(D,L-lactic-co-glycolic acid) (PLGA) nanoparticles. Other biocompatible and biodegradable polymeric materials are known in the art and can be used with the compositions and methods described herein.

Nanoparticles for use with the compositions and methods described herein range in size from about 50 nm to about 1000 nm in diameter. For use in the methods disclosed herein, the nanoparticles are typically about 600 nm or smaller in diameter. In some embodiments, the nanoparticles are about 100 to about 600 nm in diameter, about 200 to about 500 nm in diameter, or about 300 to about 450 nm in diameter.

In some examples, the TLR4 ligand is MPL. In some examples, the TLR7/TLR8 ligand is R837. Other TLR4 and TLR7/TLR8 ligands are known (for examples, see Table 2 below) and can be used with the described compositions and methods.

The dose of TLR ligand varies depending on the selected ligand. Using lower doses of TLR ligand reduces the risk of toxicity. The synergistic effect of combining two or more TLR ligands disclosed herein enables the use of lower doses of TLR ligand to achieve the same or greater enhancement of the immune response, thereby reducing the potential for toxicity. In some embodiments, the TLR4 ligand is MPL and is used at a dose of about 5 μg to about 50 μg, such as about 5, about 10, about 15, about 20, about 25, about 30, about 35, about 40, about 45 or about 50 μg. In some embodiments, the TLR7/TLR8 ligand is R837 and is used at a dose of about 10 μg to about 100 μg, such as about 10, about 20, about 30, about 40, about 50, about 60, about 70, about 80, about 90 or about 100 μg. Other TLR ligands also can be administered at the doses listed above, or any other appropriate dose.

In some embodiments, the ratio of the dose of a first TLR ligand to the dose of a second TLR ligand is approximately 1:1. In other embodiments, the ratio is about 1:2, or about 1:3, or about 1:4, or about 1:5, or about 1:6, or about 1:7, or about 1:8, or about 1:9, or about 1:10, or about 2:3, or about 2:5, or about 2:7 or about 2:9, or about 3:4, or about 3:5, or about 3:7, or about 3:8, or about 3:10, or about 4:5, or about 4:7, or about 4:9, or about 5:6, or about 5:7, or about 5:8, or about 5:9, or about 6:7, or about 7:8, or about 7:9, or about 7:10, or about 8:9, or about 9:10.

The target antigen can be any type of antigen against which an immune response is desired, including a tumor antigen or an antigen from a pathogen. The dose of antigen will vary depending on a variety of factors, including the immunogenicity of the antigen, the disease or disorder being treated, the quality of the immune response desired and the TLR ligands delivered in combination with the antigen. The synergistic effect of combining two or more TLR ligands to elicit an immune response allows the use of lower doses of antigen than would be required in the absence of the TLR ligands. In some embodiments, the antigen dose is about 0.1 μg, about 0.5 μg, about 1.0 μg, about 2.5 μg, about 5 μg, about 10 μg, about 25 μg, about 50 μg or about 100 μg.

Pathogens can include viruses, bacteria, fungi and parasites. In one embodiment, the pathogen is Bacillus anthracis, the causative agent of anthrax. In another embodiment, the pathogen is influenza, or avian influenza or H1N1 swine influenza. In another embodiment, the pathogen is HIV. In another embodiment, the pathogen is Mycobacterium tuberculosis, the causative agent of tuberculosis. In one example, the antigen is anthrax protective antigen. In another example, the antigen is avian influenza H5HA, or H1N1 swine influenza. In another example, the antigen is from Mycobacterium tuberculosis, such as CFP10, ESAT-6, Ag85 or Mtb39. In another example, the antigen is from HIV, such as gp120, gp41 or p24, or consensus sequences or gp120, gp41 or p24 or gag.

The tumor antigen can be any antigen associated with a tumor or a type of cancer. In one embodiment, the tumor antigen is a melanoma antigen, such as MAGE. In another embodiment, the tumor antigen is a breast cancer antigen, such as herceptin. In another embodiment, the tumor antigen is a prostate cancer antigen, such as PSA. In another embodiment, the tumor antigen is a pancreatic cancer antigen, such as CA19-9.

Also provided herein is a method of stimulating an immune response to an antigen in a subject. For example, the method can include administering to the subject a composition comprising the antigen, a TLR4 ligand and a TLR7/TLR8 ligand. In some examples, the antigen, TLR4 ligand and TLR7/TLR8 ligand are encapsulated by nanoparticles. In some embodiments, the TLR4 ligand is encapsulated in the same nanoparticles as the TLR7/TLR8 ligand. In other embodiments, the TLR4 ligand is encapsulated in different nanoparticles as the TLR7/TLR8 ligand. In some embodiments, the antigen is encapsulated by the same nanoparticles as the TLR ligands. In other embodiments, the antigen is encapsulated by different nanoparticles as the TLR ligands. In some embodiments, the nanoparticles are poly(lactic acid) nanoparticles, poly(glycolic acid) nanoparticles, or both. In particular examples, the nanoparticles are poly(D,L-lactic-co-glycolic acid) (PLGA) nanoparticles. As described herein, administration of both a TLR4 ligand and a TLR7/TLR8 ligand results in a synergistic stimulation of the immune response as compared to administration of a single TLR ligand.

In some embodiments, the subject has cancer. In particular examples, the cancer is melanoma, breast cancer, prostate cancer or pancreatic cancer. In some embodiments, the immune response stimulated in the subject is to a cancer antigen. In other embodiments, the subject is infected with a pathogen, such as, but not limited to Bacillus anthracis or influenza virus. In some embodiments, the immune response stimulated in the subject is to an antigen from a pathogen. In some examples, the antigen from a pathogen is anthrax protective antigen (PA) or avian influenza hemagglutinin (H5HA). In other embodiments, the subject is or has been vaccinated to prophylactically protect against disease (such as cancer or an infectious disease), and the immune response stimulated in the subject is to an antigen from the vaccine.

In some embodiments, stimulating an immune response is indicated by an increase in the production of pro-inflammatory cytokines; an increase in the number of CD8⁺ T effector cells; an increase in the number of CD8⁺ T memory cells; an increase in the number of CD4⁺ T effector or memory cells; an increase in titer of antigen-specific antibodies; an increase in antigen-specific antibody affinity; an increase in titer of neutralizing antibodies; an increase in the proliferation of naïve B cells; an increase in persistence of antigen-specific B cells; an increase in the number of germinal centers; an increase in the number of antibody secreting cells; or a combination of two or more thereof. The increase in the indicator of the immune response is relative to a control, such as a value prior to administration of the antigen or in the absence of treatment. In some embodiments, the method further comprises detecting an indicator of an immune response in a sample obtained from a subject. In some examples, the fold increase in the indicator of an immune response is at least about 2-fold, at least about 5-fold, at least about 10-fold, at least about 50-fold, or at least about 100-fold.

In some embodiments, one of the indicators of an immune response is an increase in the production of one or more pro-inflammatory cytokines, such as, but not limited to IL-6, TNF-α, IFN-α and IL-12. In some embodiments, one of the indicators of an immune response is an increase in the number of CD8⁺ T effector cells, CD8⁺ T memory cells, or both. In some embodiments, one of the indicators of an immune response is an increase in the titer and/or affinity of antigen-specific antibodies. In some embodiments, the one of the indicators of an immune response is an increase in the proliferation of B cells. Methods of detecting the above indicators of an immune response are well known in the art and are described herein. In one embodiment, the sample is a blood sample. In another embodiment, the sample is a serum sample.

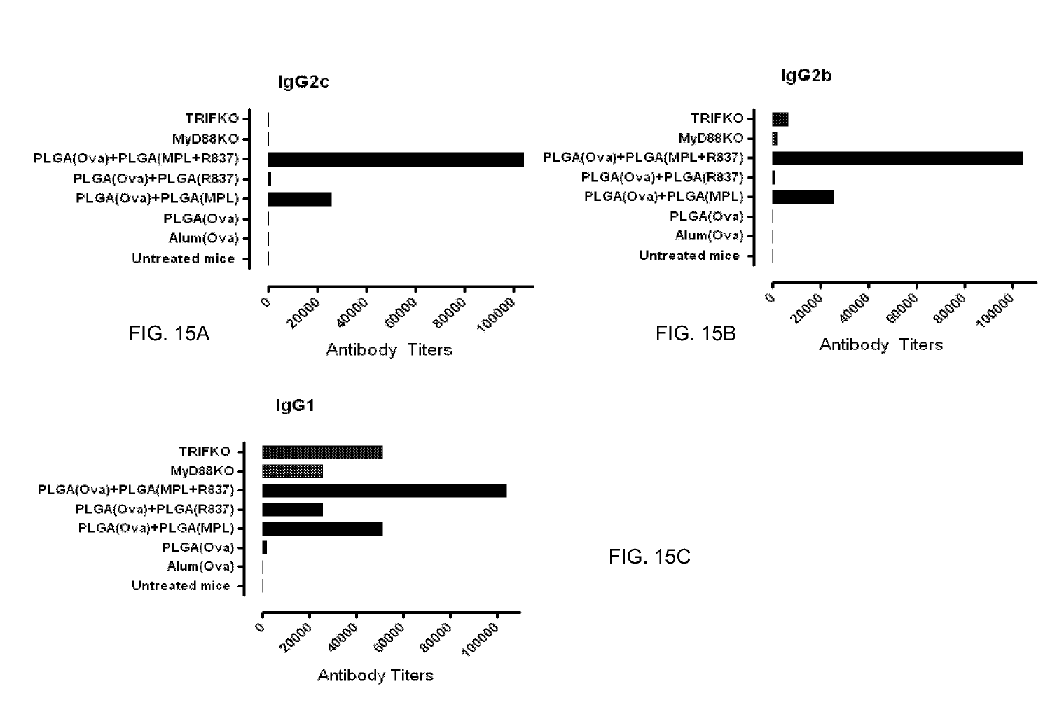

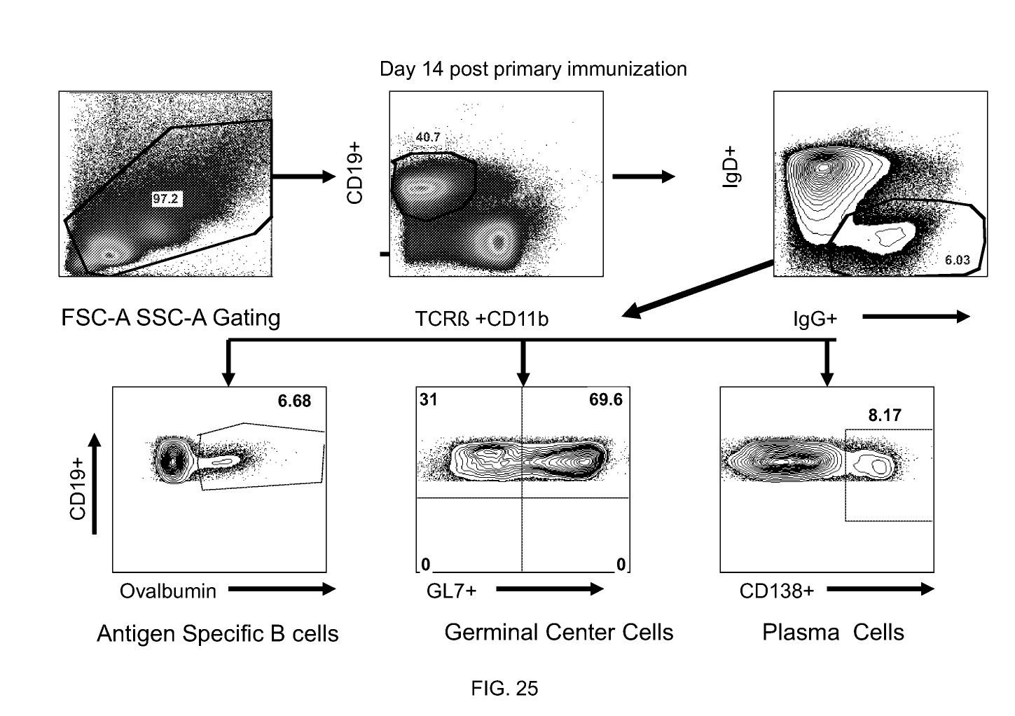

The data disclosed herein demonstrates that the synergistic induction of antibody responses induced by TLR4 ligands plus TLR7/8 ligands is dependent on MyD88 and TRIF signaling (FIG. 15 and FIG. 25). Therefore, contemplated herein is the use of any combination of TLR ligands that signal via the MyD88 and TRIF pathway. In some embodiments, the combination of TLR ligands includes TLR4 ligand and TLR9 ligand, such CpG rich oligonucleotides; or TLR3 ligand and TLR7/8 ligand; or TLR3 ligand and TLR 9 ligand.

### IV. Nanoparticles

Nanoparticles are submicron (less than about 1000 nm) sized drug delivery vehicles that can carry encapsulated drugs such as synthetic small molecules, proteins, peptides and nucleic acid based biotherapeutics for either rapid or controlled release. Nanoparticles are efficiently phagocytosed by antigen presenting cells (APCs), such as dendritic cells and macrophages, due to their pathogen-like size (typically 0.2-5 microns), as well as foreign material composition. Nanoparticles can be used as a platform technology to deliver unique combinations of antigens and adjuvants to mediate efficient prophylactic and therapeutic vaccination.

A variety of hydrophobic and hydrophilic molecules can be encapsulated in nanoparticles using processes well known in the art and described in the Examples below. Hydrophobic molecules include, but are not limited to, stimulatory molecules such as the TLR4 ligand monophosphoryl lipid A (MPL), or the small molecule TLR7/TLR8 ligand Imiquimod (R837), which can be encapsulated individually or in combination for simultaneous delivery to APCs. Hydrophilic molecules, including proteins, peptides, nucleic acids (e.g., plasmid DNA and siRNA) can also be efficiently encapsulated in nanoparticles individually or in combination for simultaneous delivery to APCs.

The nanoparticles for use with the compositions and methods described herein can be any type of biocompatible nanoparticle, such as biodegradable nanoparticles, such as polymeric nanoparticles, including, but not limited to polyamide, polycarbonate, polyalkene, polyvinyl ethers, and cellulose ether nanoparticles. In some embodiments, the nanoparticles are made of biocompatible and biodegradable materials. In some embodiments, the nanoparticles include, but are not limited to nanoparticles comprising poly(lactic acid) or poly(glycolic acid), or both poly(lactic acid) and poly(glycolic acid). In particular embodiments, the nanoparticles are poly(D,L-lactic-co-glycolic acid) (PLGA) nanoparticles.

PLGA is a FDA-approved biomaterial that has been used as resorbable sutures and biodegradable implants. PLGA nanoparticles have also been used in drug delivery systems for a variety of drugs via numerous routes of administration including, but not limited to, subcutaneous, intravenous, ocular, oral and intramuscular. PLGA degrades into its monomer constituents, lactic and glycolic acid, which are natural byproducts of metabolism, making the material highly biocompatible. In addition, PLGA is commercially available as a clinical-grade material for synthesis of nanoparticles.

Other biodegradable polymeric materials are contemplated for use with the compositions and methods described herein, such as poly(lactic acid) (PLA) and polyglycolide (PGA). Additional useful nanoparticles include biodegradable poly(alkylcyanoacrylate) nanoparticles (Vauthier et al., Adv. Drug Del. Rev. 55: 519-48, 2003). Oral adsorption also may be enhanced using poly(lactide-glycolide) nanoparticles coated with chitosan, which is a mucoadhesive cationic polymer. The manufacture of such nanoparticles is described, for example, by Takeuchi et al. (Adv. Drug Del. Rev. 47: 39-54, 2001).

Among the biodegradable polymers currently being used for human applications, PLA, PGA, and PLGA are known to be generally safe because they undergo in vivo hydrolysis to harmless lactic acid and glycolic acid. These polymers have been used in making sutures when post-surgical removal is not required, and in formulating encapsulated leuprolide acetate, which has been approved by the FDA for human use (Langer and Mose, Science 249:1527, 1990); Gilding and Reed, Polymer 20:1459, 1979; Morris, et al., Vaccine 12:5, 1994). The degradation rates of these polymers vary with the glycolide/lactide ratio and molecular weight thereof. Therefore, the release of the encapsulated drug can be sustained over several months by adjusting the molecular weight and glycolide/lactide ratio of the polymer, as well as the particle size and coating thickness of the capsule formulation (Holland, et al., J. Control. Rel. 4:155, 1986).

Nanoparticles for use with the compositions and methods described herein range in size from about 50 nm to about 1000 nm in diameter. In general, smaller nanoparticles are preferentially taken up by DCs, while larger nanoparticles are internalized by macrophages. Thus, for use in the methods disclosed herein, the nanoparticles are typically less than about 600 nm. In some embodiments, the nanoparticles are about 100 to about 600 nm in diameter. In some embodiments, the nanoparticles are about 200 to about 500 nm in diameter. In some embodiments, the nanoparticles are about 300 to about 450 nm in diameter. One skilled in the art would readily recognize that the size of the nanoparticle may vary depending upon the method of preparation, clinical application, and imaging substance used.

Various types of biodegradable and biocompatible nanoparticles, methods of making such nanoparticles, including PLGA nanoparticles, and methods of encapsulating a variety of synthetic compounds, proteins and nucleic acids, has been well described in the art (see, for example, U.S. Publication No. 2007/0148074; U.S. Publication No. 20070092575; U.S. Patent Publication No. 2006/0246139; U.S. Pat. No. 5,753,234; U.S. Pat. No. 7,081,489; and PCT Publication No. WO/2006/052285).

In some embodiments, the two or more TLR ligands are encapsulated in the same nanoparticles. In other embodiments, each TLR ligand is encapsulated in different nanoparticles. In some embodiments, the antigen is encapsulated in the same nanoparticles as one, both or all of the TLR ligands. In other embodiments, the antigen is encapsulated in different nanoparticles than the TLR ligands.

### V. Antigens

Any type of antigen, such as an antigen from a pathogen or a tumor-specific antigen, can be used with the compositions and methods described herein. The choice of antigen is determined by the type of immune response that is desired. For example, to elicit an immune response against influenza, an influenza-specific antigen is selected, such as H5HA. As another example, if an immune response against malignant melanoma is desired, a melanoma-specific antigen, such as melanoma-associated antigen (MAGE), is selected.

In some embodiments, the nanoparticles are loaded with antigen produced as a recombinant protein or peptide. In other embodiments, a plasmid encoding the selected antigen is encapsulated in the nanoparticle.

The dose of antigen will vary depending on a variety of factors, including the immunogenicity of the antigen, the disease or disorder being treated, the quality of the immune response desired and the TLR ligands delivered in combination with the antigen. The synergistic effect of combining two or more TLR ligands to elicit an immune response allows the use of lower doses of antigen than would be required in the absence of the TLR ligands. In some embodiments, the antigen dose is about 0.1 μg, about 0.5 μg, about 1.0 μg, about 2.5 μg, about 5 μg, about 10 μg, about 25 μg, about 50 μg or about 100 μg.

In some cases, the selected antigen is an antigen from a pathogen, such as a virus, bacterium, fungus or parasite. Viral pathogens include, but are not limited to retroviruses, such as human immunodeficiency virus (HIV) and human T-cell leukemia viruses; picornaviruses, such as polio virus, hepatitis A virus; hepatitis C virus, enteroviruses, human coxsackie viruses, rhinoviruses, echoviruses, and foot-and-mouth disease virus; caliciviruses, such as strains that cause gastroenteritis (e.g., Norwalk virus); togaviruses, such as alphaviruses (including chikungunya virus, equine encephalitis viruses, Sindbis virus, Semliki Forest virus, and Ross River virus) and rubella virus; flaviviruses, such as dengue viruses, yellow fever viruses, West Nile virus, St. Louis encephalitis virus, Japanese encephalitis virus, Powassan virus and other encephalitis viruses; coronaviruses, including severe acute respiratory syndrome (SARS) virus; rhabdoviruses, such as vesicular stomatitis virus and rabies virus; filoviruses, such as Ebola virus and Marburg virus); paramyxoviruses, such as parainfluenza virus, mumps virus, measles virus, and respiratory syncytial virus; orthomyxoviruses, such as influenza viruses, including swine flu and avian flu viruses; bunyaviruses, such as Hantaan virus; Sin Nombre virus, and Rift Valley fever virus, phleboviruses and Nairo viruses; arenaviruses, such as Lassa fever virus and other hemorrhagic fever viruses, Machupo virus and Junin virus; reoviruses, such as mammalian reoviruses, orbiviurses and rotaviruses; birnaviruses; hepadnaviruses, such as hepatitis B virus; parvoviruses; papovaviruses, such as papilloma viruses, polyoma viruses and BK-virus; adenoviruses; herpesviruses, such as herpes simplex virus (HSV)-1 and HSV-2, cytomegalovirus, Epstein-Barr virus, varicella zoster virus, and other herpes viruses, including HSV-6); pox viruses, such as variola viruses and vaccinia viruses; irodoviruses, such as African swine fever virus; astroviruses; and unclassified viruses (for example, the etiological agents of spongiform encephalopathies, the agent of delta hepatitis (thought to be a defective satellite of hepatitis B virus).

Bacterial pathogens include, but are not limited to Helicobacter pylori, Escherichia coli, Vibrio cholerae, Borelia burgdorferi, Legionella pneumophilia, Mycobacteria sps (such as. M. tuberculosis, M. avium, M. intracellulare, M. kansai and, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Bordetella pertussis, Shigella flexnerii, Shigella dysenteriae and Actinomyces israelli.

Fungal pathogens include, but are not limited to Cryptococcus neoformans, Histoplasma capsulatum, Coccidioides immitis, Blastomyces dermatitidis, Chlamydia trachomatis, Candida albicans. Parasitic pathogens include, but are not limited to Plasmodium falciparum, Plasmodium vivax, Trypanosoma cruzi and Toxoplasma gondii.

In one embodiment, the pathogen is HIV. HIV antigens include, but are not limited to, gag, pol, nef, vpr, gp120, gp41 and p24. In another embodiment, the pathogen is Mycobacterium tuberculosis. Tuberculosis antigens include, but are not limited to, CFP10, ESAT-6, Ag85 and Mtb39. In another embodiment, the pathogen is influenza virus. In another embodiment, the pathogen is a malaria parasite (e.g., Plasmodium falciparum or Plasmodium vivax). In another embodiment, the pathogen is Bacillus anthracis (the causative agent of anthrax). In another embodiment, the pathogen is chikungunya virus. In another embodiment, the pathogen is dengue virus. In another embodiment, the pathogen is hepatitis C virus. In another embodiment, the pathogen is SARS virus. In another embodiment, the pathogen is Ebola virus. In another embodiment, the pathogen is Lassa fever virus. In another embodiment, the pathogen is West Nile virus. In another embodiment, the pathogen is Vibrio cholerae. In another embodiment, the pathogen is Shigella flexnerii or Shigella dysenteriae.

In one example, the antigen is anthrax protective antigen (PA). In another example, the antigen is influenza antigen H5HA. In another embodiment, the antigen is from the H1N1 swine influenza virus.

In some cases, the antigen is a tumor-associated antigen. Tumor antigens are proteins that are produced by tumor cells that elicit an immune response, particularly T-cell mediated immune responses. The tumor antigen can be any tumor-associated antigen, which are well known in the art and include, for example, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. A list of exemplary tumor antigens and their associated tumors are shown below in Table 1.

TABLE 1
Exemplary tumors and their tumor antigens

| Tumor | Tumor Associated Target Antigens |
|---|---|
| Acute myelogenous leukemia | Wilms tumor 1 (WT1), preferentially expressed |
| | antigen of melanoma (PRAME), PR1, proteinase 3, |
| | elastase, cathepsin G |
| Chronic myelogenous leukemia | WT1, PRAME, PR1, proteinase 3, elastase, cathepsin G |
| Myelodysplastic syndrome | WT1, PRAME, PR1, proteinase 3, elastase, cathepsin G |
| Acute lymphoblastic leukemia | PRAME |
| Chronic lymphocytic leukemia | Survivin |
| Non-Hodgkin's lymphoma | Survivin |
| Multiple myeloma | NY-ESO-1 |
| Malignant melanoma | MAGE, MART, Tyrosinase, PRAME GP100 |
| Breast cancer | WT1, herceptin, epithelial tumor antigen (ETA) |
| Lung cancer | WT1 |
| Ovarian cancer | CA-125 |
| Prostate cancer | PSA |
| Pancreatic cancer | CA19-9, RCAS1 |
| Colon cancer | CEA |
| Renal cell carcinoma (RCC) | Fibroblast growth factor 5 |
| Germ cell tumors | AFP |

In one embodiment, the tumor antigen is a melanoma antigen, such as MAGE. In another embodiment, the tumor antigen is a breast cancer antigen, such as herceptin. In another embodiment, the tumor antigen is a prostate cancer antigen, such as PSA. In another embodiment, the tumor antigen is a pancreatic cancer antigen, such as CA19-9.

### VI. Toll-Like Receptors (TLR5) and TLR Ligands

TLR5 are a class of single membrane-spanning non-catalytic receptors that recognize structurally conserved molecules derived from microorganisms and play an important role in innate immune responses to pathogenic microorganisms. In vertebrates, TLR5 can help activate the adaptive immune system, linking innate and acquired immune responses. TLRs are a type of pattern recognition receptor that recognizes molecules evolutionarily conserved and broadly shared by pathogens, but distinguishable from host molecules. In humans, eleven TLR (identified as TLR1 to 11) have been identified thus far. TLRs function (bind to ligands) as dimers, and most form homodimers. For most TLRs, one or more specific ligands have been identified and are listed in Table 2 below. Most ligands that bind TLR7 also bind TLR8; however, some synthetic ligands bind only TLR7 or only TLR8.

TABLE 2
TLRs and known TLR Ligands

| TLR | TLR Ligand(s) |
|---|---|
| TLR1 | Multiple triacyl lipopeptides |
| TLR2 | Multiple glycolipids, lipopeptides and lipoproteins |
| | Lipoteichoic acid |
| | Peptidoglycan |
| | HSP70 |
| | Zymosan |
| TLR3 | Double-stranded RNA |
| | Poly(I:C) |
| TLR4 | LPS |
| | Monophosphoryl lipid A (MPL) |
| | Several heat shock proteins |
| | Fibrinogen |
| | Heparin sulfate fragments |
| | Hyaluronic acid fragments |
| TLR5 | Flagellin |
| TLR6 | Multiple diacyl lipopeptides |
| TLR7 | Imidazoquinolines (e.g., imiquimod and resiquimod) |
| | GU-rich single-stranded RNA, |
| | Loxoribine (a guanosine analog) |
| | Bropirime |
| TLR8 | Imidazoquinolines (e.g., imiquimod and resiquimod) |
| | GU-rich single-stranded RNA |
| | Small synthetic compounds |
| TLR9 | Unmethylated CpG DNA |
| | Hemazoin crystals |
| TLR10 | Unknown |
| TLR11 | Toxoplasma gondii profilin |
| | Uropathogenic-bacteria-derived protein |

Previous studies have shown that delivery of nanoparticles or microparticles containing antigen and a TLR ligand enhances antigen-specific immunity and T helper immune responses (Hamdy et al., J. Biomed. Mater. Res. A. 81(3):652-62, 2007; Chong et al., J. Control. Release 102(1):85-99, 2005; Heit et al., Eur. J. Immunol. 37:2063-2074, 2007). However, each of these studies used only a single TLR ligand, which was encapsulated in the same nanoparticles as the antigen. As shown herein, delivery of a combination of two different TLR ligands (along with delivery of nanoparticle-encapsulated antigen) unexpectedly results in a synergistic immune response.

In some embodiments, the combination of TLR ligands includes a TLR4 ligand and a TLR7/TLR8 ligand. In other embodiments, the combination of TLR ligands includes a TLR3 ligand and a TLR7/TLR8 ligand. In other embodiments, the combination of TLR ligands includes a TLR4 ligand and a TLR9 ligand. In other embodiments, the combination of TLR ligands includes a TLR3 ligand and a TLR9 ligand. Although exemplary combinations of TLR ligands are described herein, any combination of TLR ligands that results in a synergistic enhancement of an immune response is contemplated herein.

In some embodiments, the two TLR ligands are encapsulated in the same nanoparticles as each other. In other embodiments, the two TLR ligands are encapsulated in different nanoparticles from each other. In some embodiments, the TLR ligands include a TLR4 ligand and a TLR7/TLR8 ligand. In some examples, the TLR4 ligand is MPL and the TLR7/TLR8 ligand is imiquimod (R837). Additional combinations of TLR ligands are contemplated and include, but are not limited to a TLR 4 ligand and a TLR9 ligand; a TLR7 ligand and a TLR9 ligand; a TLR8 ligand and a TLR9 ligand; a TLR3 ligand and a TLR7 ligand; a TLR3 ligand and a TLR8 ligand; a TLR3 ligand and a TLR9 ligand; and a TLR3 ligand and a TLR4 ligand.

The dose of TLR ligand varies depending on the selected ligand. Using lower doses of TLR ligand reduces the risk of toxicity. The synergistic effect of combining two or more TLR ligands disclosed herein enables the use of lower doses of TLR ligand to achieve the same or greater enhancement of the immune response. In some embodiments, the TLR4 ligand is MPL and is used at a dose of about 5 μg to about 50 μg, such as about 5, about 10, about 15, about 20, about 25, about 30, about 35, about 40, about 45 or about 50 μg. In some embodiments, the TLR7/TLR8 ligand is R837 and is used at a dose of about 10 μg to about 100 μg, such as about 10, about 20, about 30, about 40, about 50, about 60, about 70, about 80, about 90 or about 100 μg. Other TLR ligands also can be used at the doses listed above, or any other suitable dose.

In some embodiments, the ratio of the dose of a first TLR ligand to the dose of a second TLR ligand is approximately 1:1. In other embodiments, the ratio is about 1:2, or about 1:3, or about 1:4, or about 1:5, or about 1:6, or about 1:7, or about 1:8, or about 1:9, or about 1:10, or about 2:3, or about 2:5, or about 2:7 or about 2:9, or about 3:4, or about 3:5, or about 3:7, or about 3:8, or about 3:10, or about 4:5, or about 4:7, or about 4:9, or about 5:6, or about 5:7, or about 5:8, or about 5:9, or about 6:7, or about 7:8, or about 7:9, or about 7:10, or about 8:9, or about 9:10.

Also contemplated herein is delivery of three or more TLR ligands in the same or different nanoparticles. For example, possible combinations include, but are not limited to, three of more of a TLR3 ligand, a TLR4 ligand, a TLR5 ligand, a TLR6 ligand, a TLR7 ligand, a TLR8 ligand and a TLR9 ligand.

### VII. Administration and Use of TLR Ligand-Containing Nanoparticles

Compositions that include the antigen-loaded and TLR ligand-loaded nanoparticles provided herein can be used to treat or prevent any number of infectious diseases or malignancies. Any disease or disorder that can be treated by eliciting an immune response to a specific antigen can be treated according to methods described herein. In some embodiments, a therapeutically effective amount of the compositions described herein are administered to a subject infected with a virus, such as HIV or HCV. The compositions can also be administered to a subject prophylactically to prevent infection or disease. In other embodiments, a therapeutically effective amount of the compositions described herein are administered to a subject infected with bacteria, such as Mycobacterium tuberculosis, the causative agent of tuberculosis, or Bacillus anthracis, the causative agent of anthrax. In some cases, the Mycobacterium tuberculosis is the drug-resistant form. In some embodiments, a therapeutically effective amount of the compositions described herein are administered to a subject infected with a parasite, such as a malaria parasite (e.g., Plasmodium falciparum or Plasmodium vivax). In other embodiments, a therapeutically effective amount of the compositions described herein are administered to a subject diagnosed with a tumor or cancer. In some embodiments, the tumor or cancer is melanoma, breast cancer, prostate cancer or pancreatic cancer.

The dose of TLR ligand administered to a subject varies depending on the selected ligand. Using lower doses of TLR ligand reduces the risk of toxicity. The synergistic effect of combining two or more TLR ligands disclosed herein enables the use of lower doses of TLR ligand to achieve the same or greater enhancement of the immune response. In some embodiments, the TLR4 ligand is MPL and is used at a dose of about 5 μg to about 50 μg, such as about 5, about 10, about 15, about 20, about 25, about 30, about 35, about 40, about 45 or about 50 μg. In some embodiments, the TLR7/TLR8 ligand is R837 and is used at a dose of about 10 μg to about 100 μg, such as about 10, about 20, about 30, about 40, about 50, about 60, about 70, about 80, about 90 or about 100 μg. Other TLR ligands also can be used at the doses listed above, or any other suitable dose.

In some embodiments, the ratio of the dose of a first TLR ligand to the dose of a second TLR ligand is approximately 1:1. In other embodiments, the ratio is about 1:2, or about 1:3, or about 1:4, or about 1:5, or about 1:6, or about 1:7, or about 1:8, or about 1:9, or about 1:10, or about 2:3, or about 2:5, or about 2:7 or about 2:9, or about 3:4, or about 3:5, or about 3:7, or about 3:8, or about 3:10, or about 4:5, or about 4:7, or about 4:9, or about 5:6, or about 5:7, or about 5:8, or about 5:9, or about 6:7, or about 7:8, or about 7:9, or about 7:10, or about 8:9, or about 9:10.

The compositions described herein can be administered by any route suitable for delivering the antigen- and TLR ligand-containing nanoparticles to APCs. Methods of administration include, but are not limited to, intradermal, intramuscular, transdermal, intraperitoneal, parenteral, intravenous, subcutaneous, vaginal, rectal, intranasal, inhalation, pulmonary delivery, oral or mist-spray delivery to the lungs. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local. Sterile injectable solutions are prepared by incorporating the active compounds in the required amount in the appropriate solvent with any other ingredients as required, followed by filtered sterilization.

The compositions are administered in any suitable manner, such as with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Provided herein are pharmaceutical compositions which include a therapeutically effective amount of the antigen-containing and TLR ligand-containing nanoparticles alone or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

The compositions disclosed herein can be formulated in a neutral or salt form. Pharmaceutically-acceptable salts include the acid addition salts (formed with the free amino groups of the protein) which are formed with inorganic acids such as, for example, hydrochloric or phosphoric acids, or organic acids such as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

Proper fluidity can be maintained, for example, by the use of a coating, such as lecithin, by the maintenance of the required particle size, and by the use of surfactants. In some cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Generally, dispersions are prepared by incorporating the various sterilized active ingredients into a sterile vehicle which contains the basic dispersion medium and the required components. In the case of sterile powders for the preparation of sterile injectable solutions, exemplary methods of preparation are vacuum-drying and freeze-drying techniques which yield a powder of the active ingredient plus any additional desired ingredient from a previously sterile-filtered solution thereof. The nanoparticles of the present invention may also be administered into the epidermis using the Powderject System (Chiron, Emeryville, Calif.). The Powderject delivery technique works by the acceleration of fine particles to supersonic speed within a helium gas jet and delivers pharmaceutical agents and vaccines to skin and mucosal injection sites, without the pain or the use of needles.

Administration can be accomplished by single or multiple doses. The dose administered to a subject in the context of the present disclosure should be sufficient to induce a beneficial therapeutic response in a subject over time, such as preventing or inhibiting infection by a pathogen, or inhibiting development or spread of a tumor. A therapeutically effective dose can also be determined by measuring the immune response, such as by detecting cytokine expression or T cell responses. The dose required will vary from subject to subject depending on the species, age, weight and general condition of the subject, the severity of the disease or disorder being treated, the particular composition being used and its mode of administration. An appropriate dose can be determined by one of ordinary skill in the art using only routine experimentation. In some cases, it will be desirable to have multiple administrations of the compositions, particularly when used as vaccines. Typically, a vaccine, which can be used to elicit both prophylactic and therapeutic responses, is administered in one, two, three, four, five or six does. The compositions will normally be administered at approximately two to twelve week intervals. In some cases, the compositions are administered at approximately 4-6 month intervals. Periodic boosters at intervals every 1-10 years, such as one, two, three, four, five, six, seven, eight, nine or ten years, can be administered to maintain protective levels of the antibodies.

### VIII. Methods of Detecting an Immune Response

In some embodiments of the methods disclosed herein, the methods include detection of particular indicators of an immune response. Such indicators include, but are not limited to, an increase in the production of pro-inflammatory cytokines; an increase in the number of CD8⁺ T effector cells; an increase in the number of CD8⁺ T memory cells; an increase in titer of antigen-specific antibodies; an increase in antigen-specific antibody affinity; and an increase in the proliferation of naïve B cells. The increase in the indicator of the immune response is relative to a control, such as a value prior to administration of the antigen or in the absence of treatment. In some examples, the fold increase in the indicator of an immune response is at least about 2-fold, at least about 5-fold, at least about 10-fold, at least about 50-fold, or at least about 100-fold.

Methods of detecting an increase in the production of cytokines, methods of detecting an increase in the number of a particular type of immune cell, methods of detecting activation of particular types of immune cells, and methods of detecting an increase in antibody titer and affinity are well known in art and can be carried out by one of ordinary skill in the art. Generally, the methods comprise immunological detection of the specific cytokines or antibody isotype of interest, or immunologic detection of specific markers on the cell type of interest. For evaluating binding affinity of antibody, standard binding assays can be employed. Examples of assays that can be used to detect indicators of an immune response are discussed below.

For cytokine expression, the choice of assay depends on the sample being tested. To detect cytokine expression of a particular cell type in vivo, intracellular cytokine staining can be performed by isolating the cell and detecting the cytokine of interest using an appropriate antibody linked to a fluorophore, and subjecting the sample to FACS analysis. Alternatively, to detect cytokine expression ex vivo, the cell or cells of interest are isolated and cultured for an appropriate amount of time to allow for release of cytokines into the media. The supernatant is collected and cytokines present in the media can be detected using an immunological assay, such as ELISA or Western blot.

In some embodiments, the cells of interest are PBMCs. In other embodiments, a single cell type is desired, such as, but not limited to DCs, T cells or B cells. PBMCs can be isolated using any technique known in the art. For example, whole blood can be obtained from a subject and PBMCs enriched using a sucrose density gradient. Specific cell types can be further isolated using antibodies directed against specific cell surface markers of the desired cell type. The antibodies can be conjugated to a substrate, such as magnetic beads, to aid in separation of the cells. For example, to isolate B cells, an anti-CD19 antibody conjugated to magnetic beads can be used. For T cells, antibodies specific for CD4 or CD8 can be used. For DCs, antibodies specific for CD11c can be used.

T cell numbers and B cell numbers can be evaluated by any suitable assay known in the art, such as, for example, by FACS analysis using antibodies specific for T cell markers (e.g., CD4, CD8) or B cell markers (e.g., CD19). For example, PBMCs can be isolated from a subject and the number of T cells determined by staining for CD8 using an antibody conjugated to a fluorophore and subjecting the cell sample to FACS. Activation of T cells can also be evaluated by FACS analysis by detecting intracellular IFN-γ. B cells also can be quantified by FACS analysis using a B cell-specific marker, such as CD19.

Antibody titers in a subject can be evaluated by obtaining a serum sample and detecting specific antibody isotypes using an ELISA. To differentiate among different isotypes, antibodies that specifically recognize the isotype are used in the ELISA. Alternatively, total IgG can be evaluated using an antibody that recognizes all types of IgG (such as IgG₁, IgG₂ₐ, IgG_{2b} and IgG₃).

Antibody affinity can be determined by obtaining a serum sample from a subject an evaluating affinity of the antibodies in the sample to a selected antigen (i.e., the antigen used by immunization). Antibody affinity can be evaluated using any method known in the art, such as by competitive radioimmunoassay, Scatchard analysis or surface plasmon resonance (such as by using the BIOCORE™ protein characterization system).

Detecting an increase in proliferation of a cell type, such as B cells, can be accomplished, for example, by isolating B cells from a subject and detecting incorporation of ³H-thymidine in the B cells cultured ex vivo. Incorporation of ³H-thymidine indicates the cells are undergoing cell division. Detection and quantitation of the radioisotope incorporated in the cells can be achieved using a scintillation counter.

Although the above methods can be used to detect indicators of an immune response, one of skill in the art will recognize that additional suitable methods are available and can be used in conjunction with the methods provided herein.

The following examples are provided to illustrate certain particular features and/or embodiments. These examples should not be construed to limit the disclosure to the particular features or embodiments described.

### EXAMPLES

### Example 1

### Single and Double Emulsion Techniques for Encapsulation of Proteins in PLGA Nanoparticles

Encapsulation of TLR ligands was achieved by a one step emulsion and solvent evaporation technique. Briefly, the TLR4 ligand MPL (Avanti Lipids, Alabaster, Ala.) was dissolved in chloroform at 5 mg/ml and the TLR7/TLR8 ligand Imiquimod (R837) (Invivogen, San Diego, Calif.) was dissolved at 10 mg/ml in DMSO with heating to enhance solubility. MPL (0.5 ml) at 5 mg/ml was added to 200 mg of PLGA polymer (RG502H, Boehringer Ingelheim, Germany) dissolved in 2.0 ml of dichloromethane. For particles containing both MPL and R837, 0.5 ml of 5 mg/ml Imiquimod in DMSO was added to the mixture of PLGA and MPL. The organic phase containing PLGA with or without MPL and R837 was homogenized with 15 ml of a 5% wt/v poly(vinyl alcohol) (PVA) solution for 2 minutes at room temperature with a Powergen 500 homogenizer (Fisher Scientific) using speed setting 6. The oil in water emulsion (O/W) was then added to 85 ml of a 5% wt/v solution of PVA surfactant (to evaporate the organic solvent) for 4 hours at room temperature in a fume hood. The nanoparticles formed were centrifuged at 3500×g for 20 minutes and washed with 50 ml of deionized water three times to remove excess PVA and any residual solvent. The nanoparticles were then frozen at −80° C. and lyophilized using a Freezone 2.5 bench top lyophilizer (Labconco, Kansas City, Mo.).

For double emulsion processes to encapsulate recombinant proteins, 100 μl of protein solution (ovalbumin (Ova) at 100 mg/ml; anthrax protective antigen (PA) at 15 mg/ml; or avian flu specific hemagglutinin protein (HA) at 15 mg/ml) in the aqueous phase (PBS+0.5% PVA as an excipient), was homogenized with 10% wt/v PLGA solution (200 mg in 2 ml) in dichloromethane for 1.5 minutes with the Powergen homogenizer at speed 5. The water in oil emulsion (W/O) was then added to 15 ml of a 5% wt/v solution of PVA for the second emulsion step identical to the single emulsion process described above, at speed 5. The water in oil in water (W/O/W) double emulsion was then subjected to solvent evaporation for 4 hours at room temperature to generate the protein encapsulated nanoparticles. The nanoparticles formed were centrifuged at 3500×g for 20 minutes and washed with 50 ml of deionized water 3 times to remove excess PVA and any residual solvent. The nanoparticles were then frozen at −80° C. and lyophilized using a Freezone 2.5 bench top lyophilizer (Labconco, Kansas City, Mo.).

Table 3 summarizes the characteristics of the PLGA nanoparticles encapsulating the protein antigens Ova, PA and HA, as well the TLR ligands MPL and/or R837. MPL-containing PLGA nanoparticles were used at a theoretical loading of 12.5 μg of MPL/mg of formulation (100% of target load), whereas R837 was characterized by UV-Vis spectrophotometry. Antigens and TLR ligands were extracted from PLGA nanoparticles by alkaline extraction or DMSO dissolution (described below). Sizing of the nanoparticles was conducted using a dynamic light scattering based sizer (90PLUS) from Brookhaven Instruments (Holtsville, N.Y.). Sizes are represented as the volume average size distribution means that have been averaged from individually synthesized batches on multiple days indicating the reproducibility of the formulation. Antigen and R837 encapsulation was analyzed using the techniques listed below.

TABLE 3
Sizing of antigen and TLR ligand containing nanoparticles

| | | | Target | Percent | TLR | Percent |
|---|---|---|---|---|---|---|
| | Average | Protein | Loading | Loading | Loading | Loading |
| Formulation | Size (nm) | Loading | μg/mg | Efficiency | μg/mg | Efficiency |
| PLGA (Blank) | 341.9 | | | | | |
| PLGA (Ova) | 358.7 | 40.6 | 50 | 81.2 | | |
| PLGA (PA) | 322.1 | 11.9 | 15 | 79.2 | | |
| PLGA (HA) | 442.0 | 11.4 | 15 | 75.9 | | |
| PLGA (MPL) | 384.1 | | | | 12.5 | 100 |
| PLGA (R837) | 341.4 | | | | 20.8 | 83.2 |
| PLGA (MPL + R837) | 314.7 | | | | 22.7 | 90.9 |

### Alkaline Extraction Method

Approximately 5 mg of R837 encapsulating formulation was hydrolyzed overnight at 37° C. in 1 ml of 0.1N NaOH containing 2% SDS. R837 absorbance was recorded at 323 nm and a standard curve established with increasing concentrations of soluble R837. R837 encapsulation efficiencies were calculated from the standard curves using hydrolyzed MPL-containing PLGA nanoparticles for background subtraction at 323 nm for the MPL and R837 encapsulated formulations. Antigen loading was estimated using similar procedures by hydrolyzing antigen-containing PLGA formulations in 0.1 N NaOH containing 2% SDS. Protein concentrations were estimated using a standard bicinchoninic acid (BCA) assay for protein estimation (Pierce Biotechnologies, Rockford, Ill.) using soluble ovalbumin-based standard concentrations.

### DMSO Dissolution Method

Approximately 10 mg of antigen or R837 encapsulating formulations were dissolved in 0.5 ml of anhydrous DMSO by incubating at room temperature for at least 30 minutes. Intermittent high speed vortexing and bath sonication was used to aid in the dissolution of the polymer matrix. Once the solution appeared clear, 0.5 ml of DMSO containing the dissolved polymer and encapsulated protein or R837 was diluted 1:10 in 0.05% NaOH containing 0.5% SDS. The resulting clear solution was either used in a BCA assay for protein estimation or used for UV-Vis absorbance at 323 nm for R837 loading estimation. Standard curves were generated with protein and R837 in solutions of similar proportions of DMSO and NaOH/SDS.

Both alkaline encapsulation and DMSO dissolution yielded closely matching loading levels of encapsulated molecules confirming the loading efficiencies.

### Example 2

### Intracellular Delivery of PLGA Nanoparticle-Encapsulated Ovalbumin to Dendritic Cells in Vitro

Ovalbumin was labeled with Alexa488 fluorophore using conjugation techniques as described by the reagent supplier (Invitrogen, Carlsbad, Calif.). Alexa488-labeled ovalbumin was encapsulated in PLGA nanoparticles as described in Example 1 using a double emulsion/solvent evaporation technique. C57BL6 mice were injected with 20 μg recombinant Flt-3 growth factor protein per day for 9 days. Flt-3 expanded splenocytes were processed and frozen for experimental use. CD11c⁺ dendritic cells from the frozen splenocytes were enriched using a magnetic bead based positive selection isolation technique.

Enriched CD11c⁺ dendritic cells were pulsed with soluble or PLGA encapsulated Alexa488-labeled ovalbumin for 3 hours in RPMI (10% FBS, 1% penicillin/streptomycin, 1% sodium pyruvate, 1% non-essential amino acids and 1% HEPES buffer). Flow cytometry was used to detect the presence of Alexa488-labeled ovalbumin in CD11c⁺ DCs. The results demonstrated that PLGA-encapsulated protein was taken up more efficiently by both lymphoid and myeloid DC subsets compared to soluble proteins.

### Example 3

### Co-Delivery of PLGA Nanoparticle-Encapsulated TLR Ligands and PLGA Nanoparticle-Encapsulated Antigen

C57BL6 mice were subcutaneously injected at the base of the tail with 50 μg of Alexa488-labeled ovalbumin encapsulated in PLGA nanoparticles. Some mice were also injected with PLGA nanoparticles containing either MPL, R837, or both. The doses of MPL and R837 were approximately 36 and 60 μg, respectively. Draining inguinal lymph nodes were collected at 24 hours post immunization and digested using collagenase type IV enzyme for 30 minutes at 37° C. Cells obtained from the lymph nodes were passed through a 70 μM. cell strainer (BD Biosciences) and washed with 2 mM. EDTA-containing PBS buffer. Total cell number was determined and the cells were stained for several cell surface markers to define particular DC populations. Cell populations were defined as shown in Table 4.

TABLE 4
Dendritic Cell Populations by Surface Marker Expression

| Cell Population | Cell-Surface Markers |
|---|---|
| Conventional DC | CD11c⁺ |
| Plasmacytoid DC | CD11c⁺, PDCA-1⁺ |
| Dermal DC | CD11c⁺, DEC205^{int}, CD8α⁻ |
| Langerhans DC | CD11c⁺, DEC205⁺, CD8α⁻ |
| Myeloid DC | CD11c⁺, DEC205⁻, CD8α⁻ |
| Lymphoid DC | CD11c⁺, DEC205⁺, CD8α⁺ |

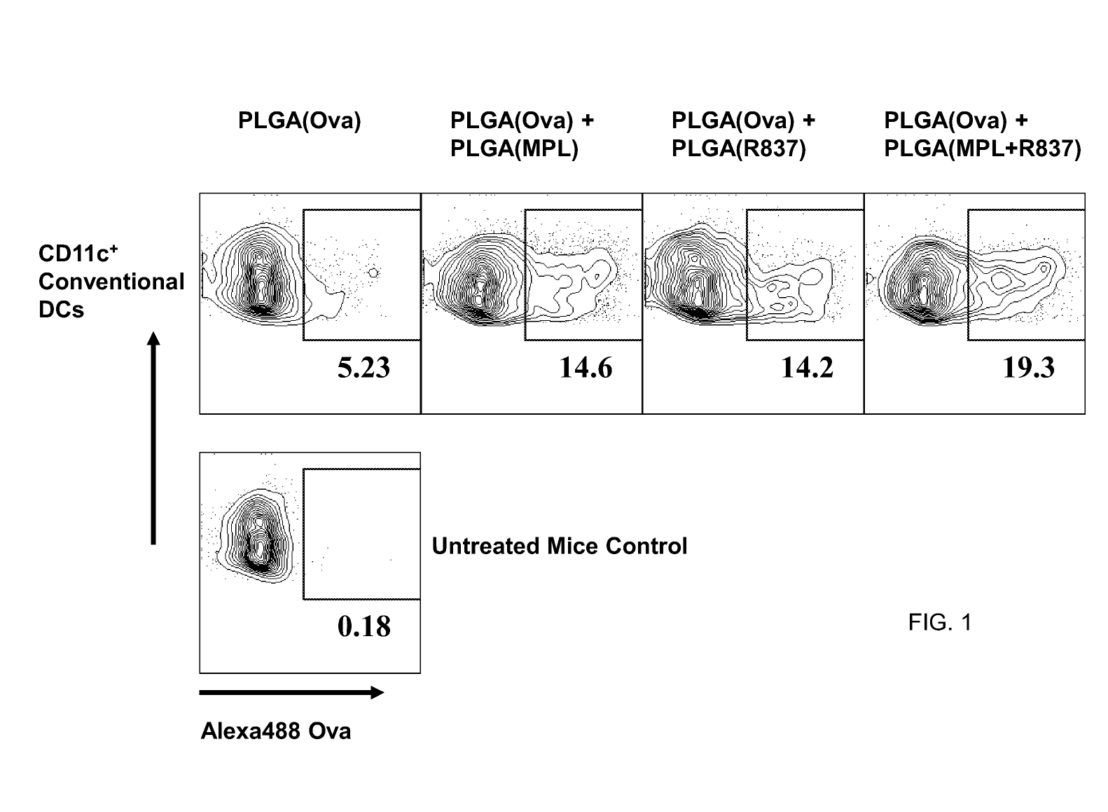

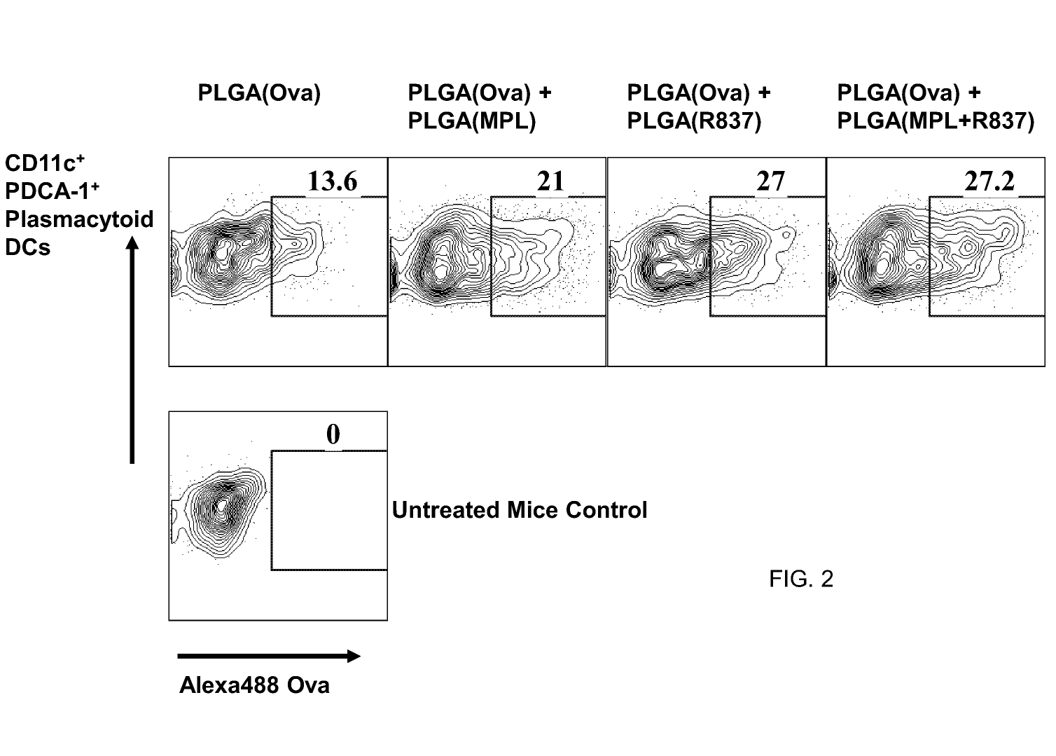

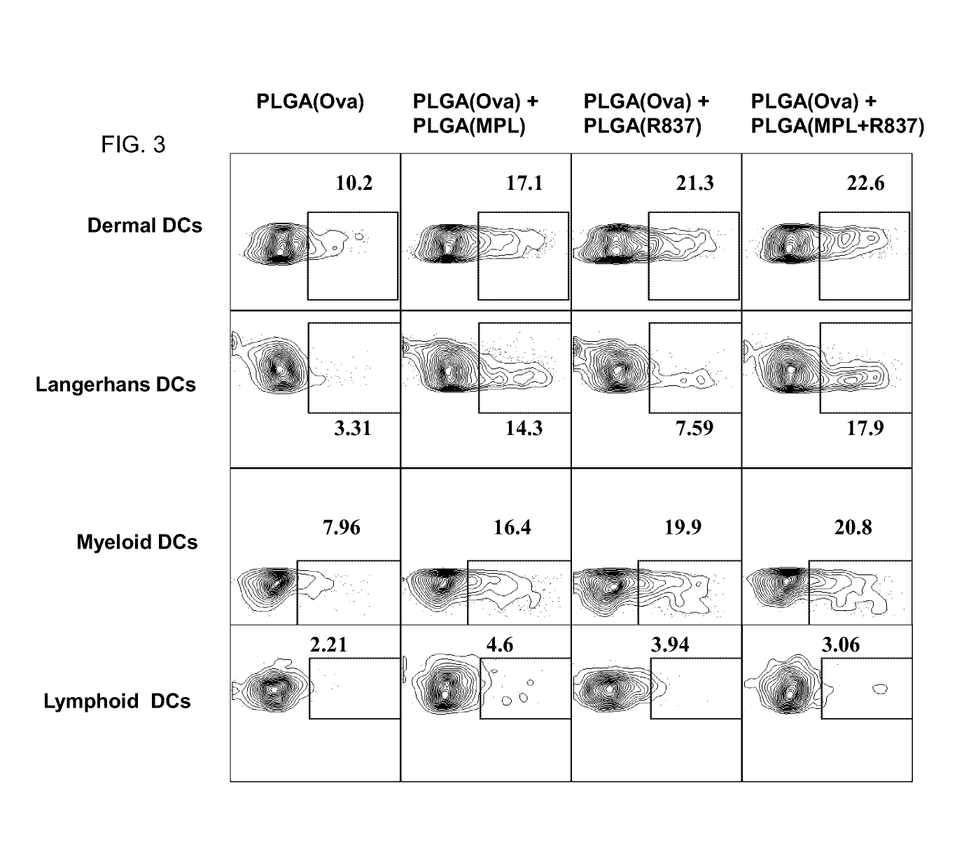

Each cell population was evaluated for uptake of Alexa488-Ova by FACS. As shown in FIG. 1, conventional DCs isolated from lymph nodes exhibited increased uptake of Alexa488-labeled ovalbumin when exposed to nanoparticles containing TLR4 ligand MPL, TLR7/TLR8 ligand R837, or both TLR ligands. Similar results were obtained in each DC population (see FIGS. 2 and 3). The significant enhancement of Alexa488-Ova uptake after treatment with TLR ligands indicates an early innate immune mechanism involving dendritic cell subsets that may enhance the subsequent CD8⁺ T cell and B cell responses by presenting an increased amount of antigen to these adaptive immune cells.

### Example 4

### Delivery of MPL and R837 Synergistically Enhances Pro-Inflammatory Cytokine Production of DCs

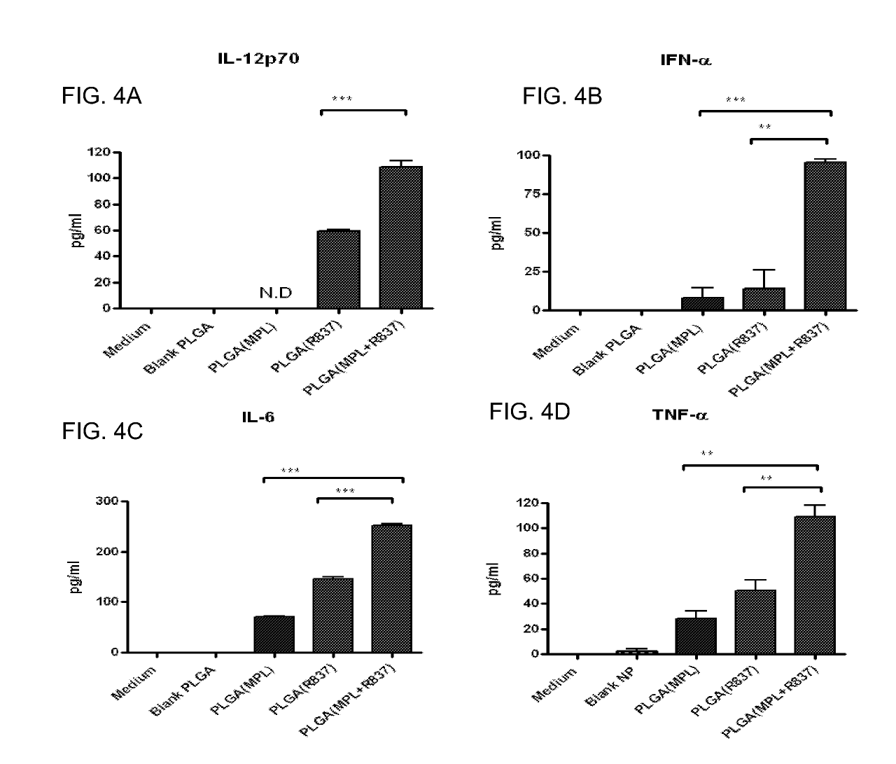

CD11c⁺ DCs were enriched from Flt-3 expanded splenocytes using magnetic bead based positive selection. Enriched DCs (1×10⁶) were cultured in 48-well plates and treated for 24 hours with either soluble ovalbumin or PLGA-encapsulated ovalbumin, in the presence or absence of soluble or PLGA-encapsulated MPL, R837 or both MPL and R837 (see FIGS. 4A-4D). The doses of MPL and R837 were approximately 36 and 60 μg, respectively. The supernatants were collected and cytokine ELISAs were performed to quantify the amount of innate immune stimulation mediated by soluble and PLGA-encapsulated TLR ligands. Combined delivery of MPL and R837 to DCs led to synergistic enhancement in the production of IL-12p70, IFN-α, IL-6 and TNF-α in vitro.

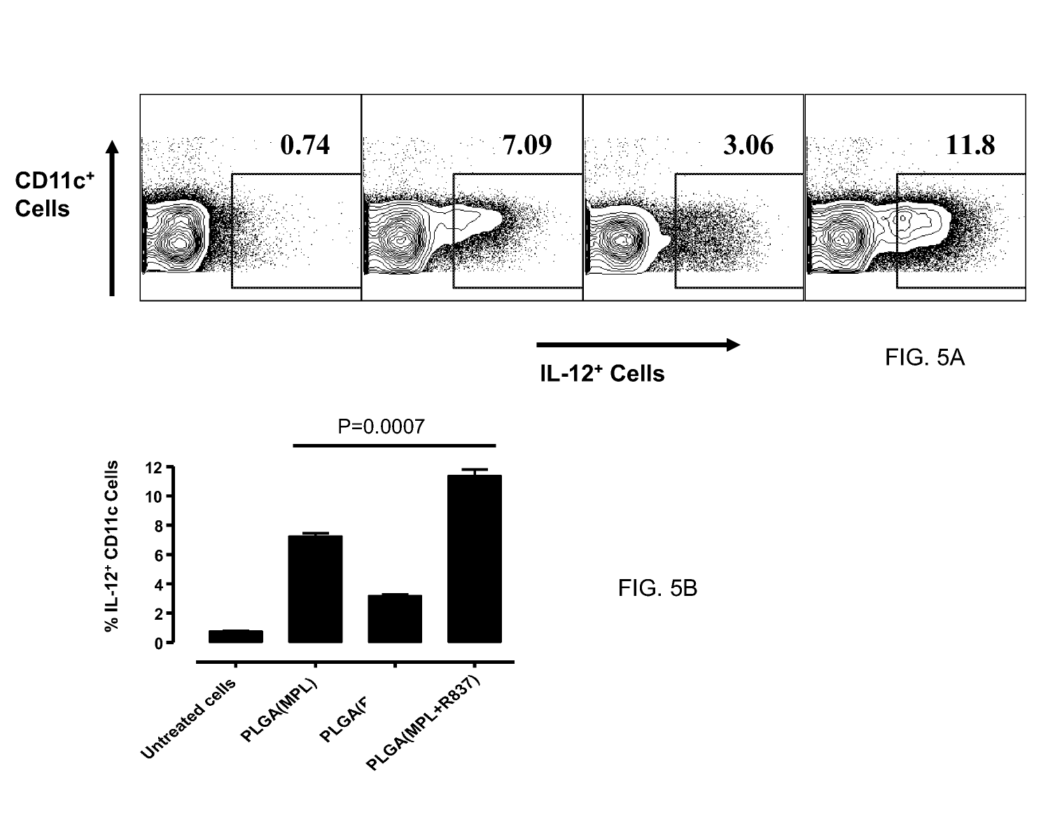

Production of IL-12p40 by Flt-3 expanded splenocyte-derived CD11c⁺ DCs was also analyzed by intracellular cytokine staining after incubation with TLR ligand-containing nanoparticles. Enriched DCs (1×10⁶) were cultured in 48-well plates for 8 hours with soluble or PLGA-encapsulated ovalbumin in the presence or absence of soluble or PLGA-encapsulated TLR ligand(s) (see FIG. 5). Brefeldin A (Sigma Aldrich), a protein transport inhibitor, was added to the DC cultures with nanoparticles. The cells were stained using antibodies specific for the cell markers CD11c (BD Biosciences), PDCA-1 (E-Biosciences) and IL-12p40/70 (BD Biosciences). FIG. 5 shows the FACS plots of CD11c⁺ DCs that are positive for IL-12p40/70 after 8 hours of stimulation. Treatment with the combination of TLR ligands MPL and R837 led to a synergistic enhancement of IL-12p40/70 cytokine production from CD11c⁺ DCs in vitro.

### Example 5

### Delivery of PLGA-Encapsulated TLR Ligand MPL Leads to Enhanced Stimulation of Effector CD8⁺ T Cell Responses in Vivo Compared to Soluble MPL

To evaluate the effect of PLGA-encapsulated MPL on CD8⁺ T cell response kinetics, C57BL6 mice were treated with either (1) 50 μg of soluble ovalbumin and 36 μg of soluble MPL; (2) 50 μg of ovalbumin and 36 μg of MPL encapsulated in the same nanoparticles; or (3) 50 μg of ovalbumin and 36 μg of MPL encapsulated in different nanoparticles. Mice were bled via the lateral tail vein on days 0, 7, 14, 28, 35, 42, 49 and 63 after treatment, and peripheral blood mononuclear cells (PBMCs) were enriched using HISTOPAQUE™ sucrose density gradient (Sigma Aldrich, St Louis, Mo.). Cells were stimulated with ovalbumin-specific class I peptide (SIINFEKL; SEQ ID NO: 1) at a concentration of 5 μg/ml, along with brefeldin A at concentration of 5 μg/ml, for 6 hours at 37° C. Cells were stained for CD8α (BD Biosciences) and intracellular cytokines IFNγ, TNFα and IL-2.

The results demonstrated that delivery of protein antigen and TLR4 ligand MPL (Avanti Lipids, Alabaster, Ala.) in two separate particles results in a greater number of effector CD8⁺ T cells and more robust effector responses compared to co-encapsulation of antigen and MPL in one particle, or compared to delivery of soluble antigen and soluble MPL. In addition, a very robust secondary memory response was observed after a boost immunization (on day 35) with the same formulations.

### Example 6

### Delivery of PLGA-Encapsulated TLR Ligand R837 Leads to Enhanced Stimulation of Effector CD8⁺ T Cell Responses in Vivo Compared to Soluble R837

To evaluate the effect of PLGA-encapsulated R837 on CD8⁺ T cell response kinetics, C57BL6 mice were treated with either (1) 50 μg of soluble ovalbumin and 60 μg of soluble R837; (2) 50 μg of ovalbumin and 60 μg of R837 encapsulated in the same nanoparticles; or (3) 50 μg of ovalbumin and 60 μg of R837 encapsulated in different nanoparticles. Mice were bled via the lateral tail vein on days 0, 7, 14, 28, 35, 42, 49 and 63 after treatment, and peripheral blood mononuclear cells (PBMCs) were enriched using the HISTOPAQUE™ sucrose density gradient (Sigma Aldrich, St Louis, Mo.). Cells were stimulated with ovalbumin-specific class I peptide (SIINFEKL; SEQ ID NO: 1) at a concentration of 5 μg/ml, along with brefeldin A at concentration of 5 μg/ml, for 6 hours at 37° C. Cells were stained for CD8α (BD Biosciences) and intracellular cytokines IFNγ, TNFα and IL-2.

The results demonstrated that delivery of protein antigen and TLR7 ligand Imiquimod (R837) (Invivogen, San Diego, Calif.) in the same nanoparticle is important for mediating the adjuvant effects of R837 on CD8⁺ T cell responses. In addition, a robust secondary memory response was observed after a boost immunization (on day 35) with the same formulations.

### Example 7

### Co-Delivery of Nanoparticle-Encapsulated TLR Ligand with Nanoparticle-Encapsulated Antigen Mediates Synergistic Enhancement in CD8⁺ T Cell and Memory CD4⁺ T Responses in Vivo

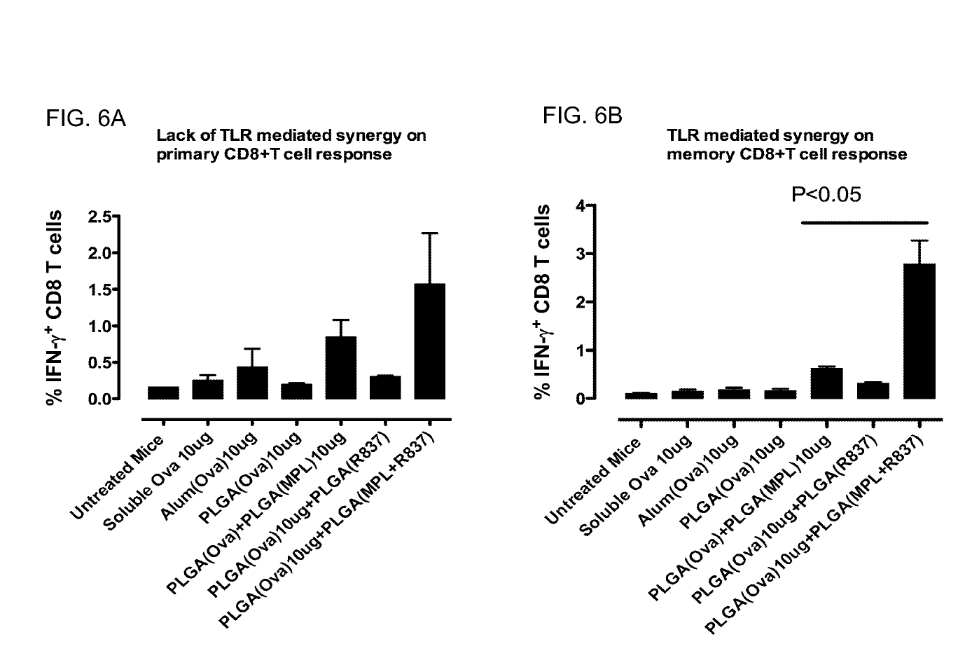

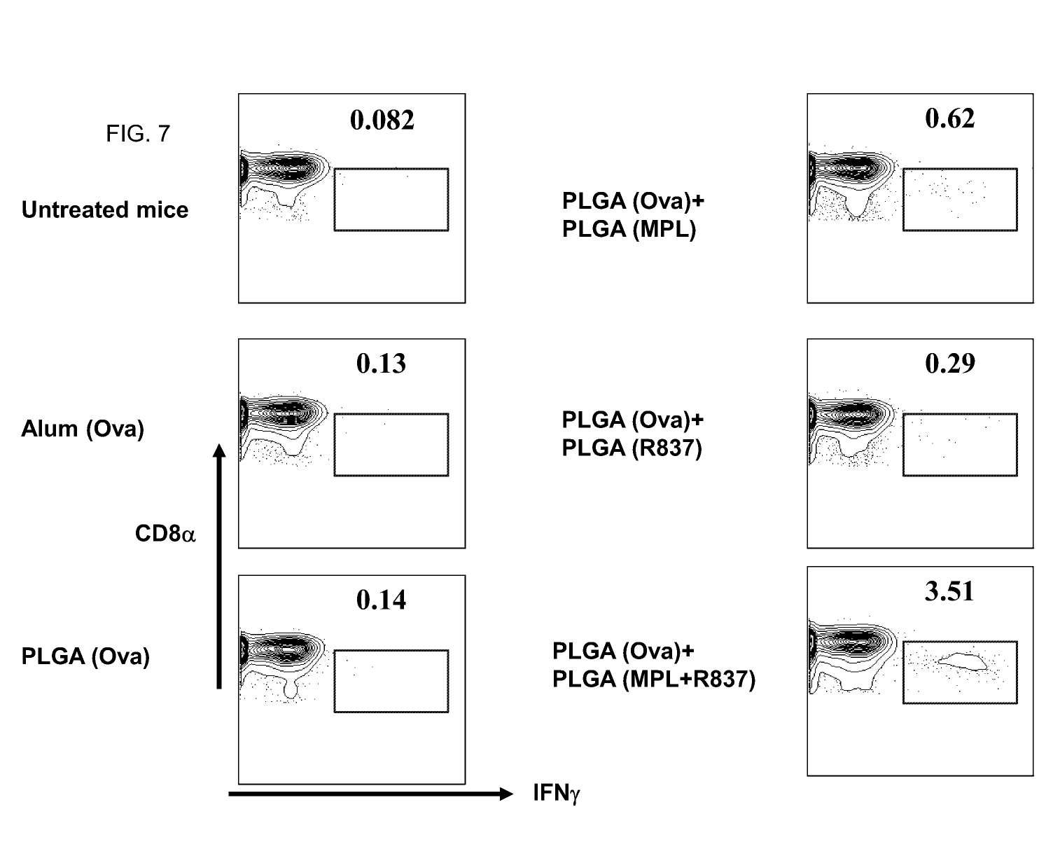

To determine whether delivery of PLGA nanoparticles containing both MPL and R837 results in a synergistic CD8⁺ T cell response, C57BL6 mice were immunized with 10 μg of soluble ovalbumin or ovalbumin encapsulated in PLGA nanoparticles. Some treatment groups were also treated with nanoparticles containing MPL, nanoparticles containing R837 or nanoparticles containing both MPL and R837. The doses of MPL and R837 were approximately 36 and 60 μg, respectively. Primary and memory CD8⁺ T cell responses were evaluated seven days after primary and secondary immunizations. Briefly, peripheral blood cells (PBCs) were enriched using sucrose density gradient separation (HISTOPAQUE™, Sigma Aldrich, Mo.) and cultured with an ovalbumin-specific MHC class I restricted peptide (SIINFEKL; SEQ ID NO: 1) for restimulation ex vivo in the presence of brefeldin A (5 μg/ml). Stimulated cells were stained for intracellular cytokines FIG. 6 shows the frequencies of CD8⁺ T cells that stained positive for IFNγ, the production of which is an indicator of a CD8⁺ T cell effector response. At sub-optimal antigen doses, combined delivery of TLR ligands MPL and R837 resulted in a synergistic enhancement of memory CD8⁺ T cell generation in vivo compared to immunization with MPL or R837 alone. FIG. 7 shows representative FACS plots from one mouse per treatment condition for the data summarized in FIG. 6.

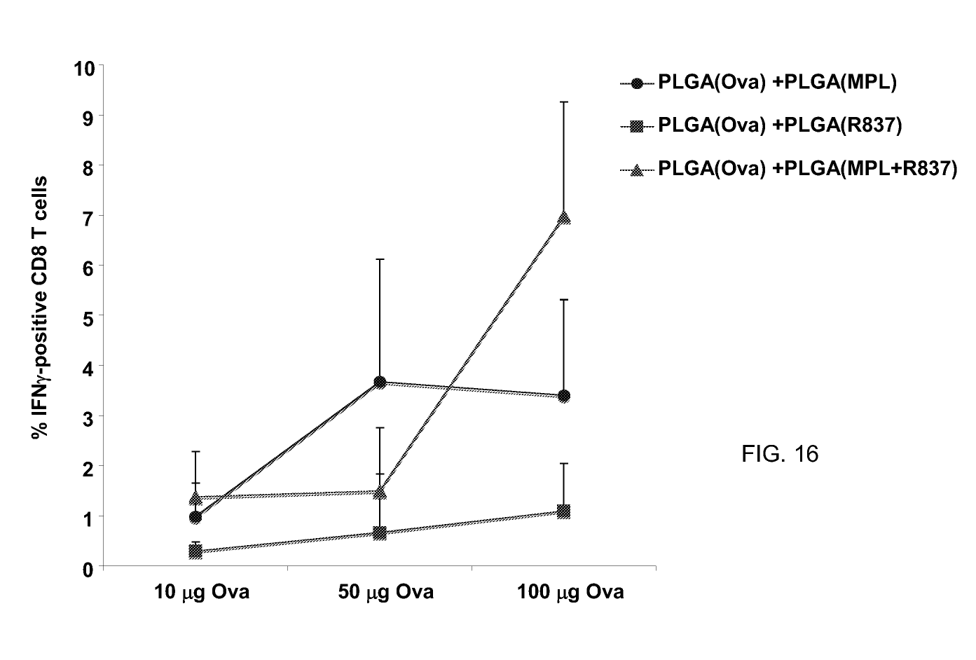

CD8⁺ T cell responses were further evaluated in response to higher antigen doses using the method described above. For this experiment, C57BL6 mice were immunized with 10 μg, 50 μg, or 100 μg of ovalbumin encapsulated in PLGA nanoparticles, in combination with nanoparticles containing MPL, R837 or both MPL and R837. The doses of MPL and R837 were approximately 36 and 60 μg, respectively. As shown in FIG. 16, the magnitude of the CD8⁺ T cell response, as measured by IFNγ production, increased as the dose of antigen was increased. In accordance with the other findings described herein, delivery of nanoparticles containing both MPL and R837 resulted in a significantly greater CD8⁺ T cell response relative to delivery of nanoparticles containing a single TLR ligand.

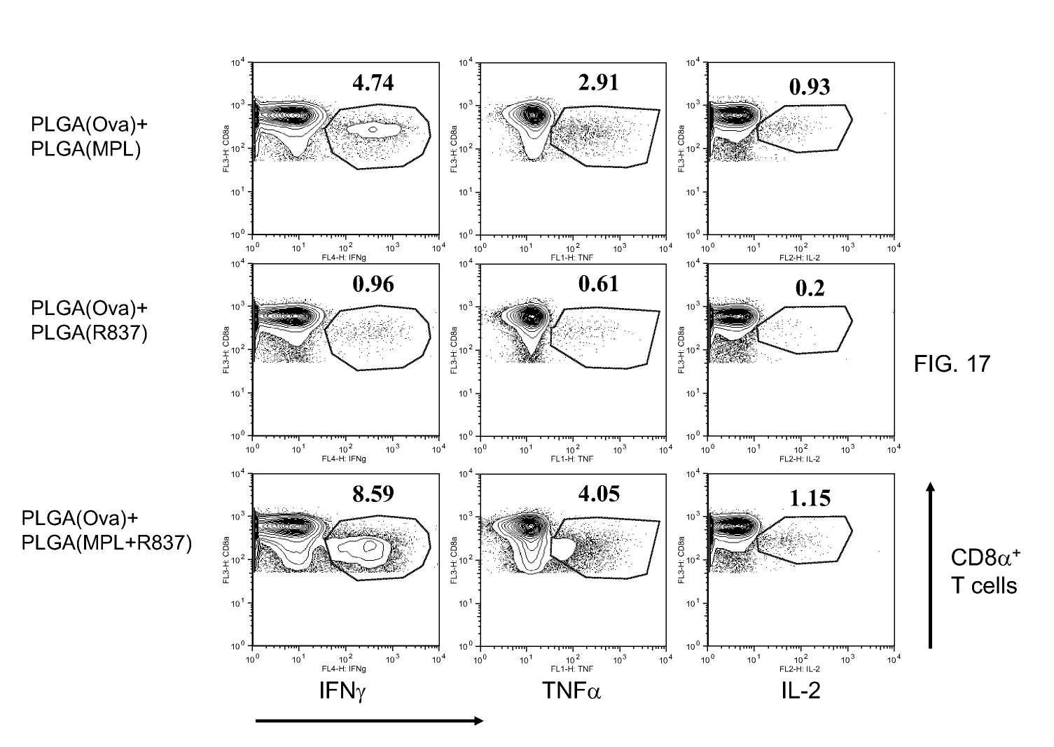

To further evaluate CD8⁺ T cell responses in vivo in response to nanoparticles containing both MPL and R837, C57BL6 mice were immunized with an optimal dose (100 μg) of ovalbumin-containing PLGA nanoparticles, in combination with nanoparticles containing MPL, R837 or both MPL and R837. The doses of MPL and R837 were approximately 36 and 60 μg, respectively. CD8⁺ T cell responses were analyzed in enriched PBMCs from mouse blood obtained at day 7 after primary immunization. FACS analysis was performed on the enriched cells to quantify CD8⁺ T cells expressing IFN-γ, TNF-α and IL-2. As shown in FIG. 17, delivery of nanoparticles containing both MPL and R837 led to a synergistic increase in cytokine production as compared to delivery of nanoparticles containing a single TLR ligand. The results demonstrate that the combination of TLR ligands (MPL and R837) leads to multifunctional cytokine producing CD8⁺ T cell responses in vivo.

To further evaluate cytokine production of CD8⁺ T cells in response to the combination of TLR ligands, C57BL6 mice were immunized with 10 μg, 50 μg, or 100 μg of ovalbumin encapsulated in PLGA nanoparticles, in combination with nanoparticles containing both MPL and R837. The doses of MPL and R837 were 36 μg and 60 μg, respectively. CD8⁺ T cell responses were evaluated at day 7 post immunization. Combinations of IFNγ, TNF-α and IL-2 producing CD8⁺ T cell populations were analyzed using FlowJO software (TreeStar Inc., Ashland, Oreg.). Proportions of triple cytokine producing (IFNγ+TNF-α+IL-2), double cytokine producing (any combination of IFNγ+TNF-α, IFNγ+IL-2, or IFNγ+TNFα), and single cytokine producing (IFNγ or TNF-α or IL-2) CD8⁺ T cells are shown in Table 5.

TABLE 5
Percentage of single, double and triple
cytokine-producing CD8⁺ T cells

| | % Single | % Double | % Triple |
|---|---|---|---|
| Ova (μg) | Cytokine | Cytokine | Cytokine |
| 10 | 35 | 56 | 9 |
| 50 | 44 | 46 | 10 |
| 100 | 52 | 42 | 6 |

These data demonstrate that delivery of nanoparticles containing the combination of TLR ligands MPL and R837 leads to multifunctional cytokine producing CD8⁺ T cell responses with substantially identical proportions of triple, double or single cytokine producing cells at both suboptimal and optimal antigen doses.

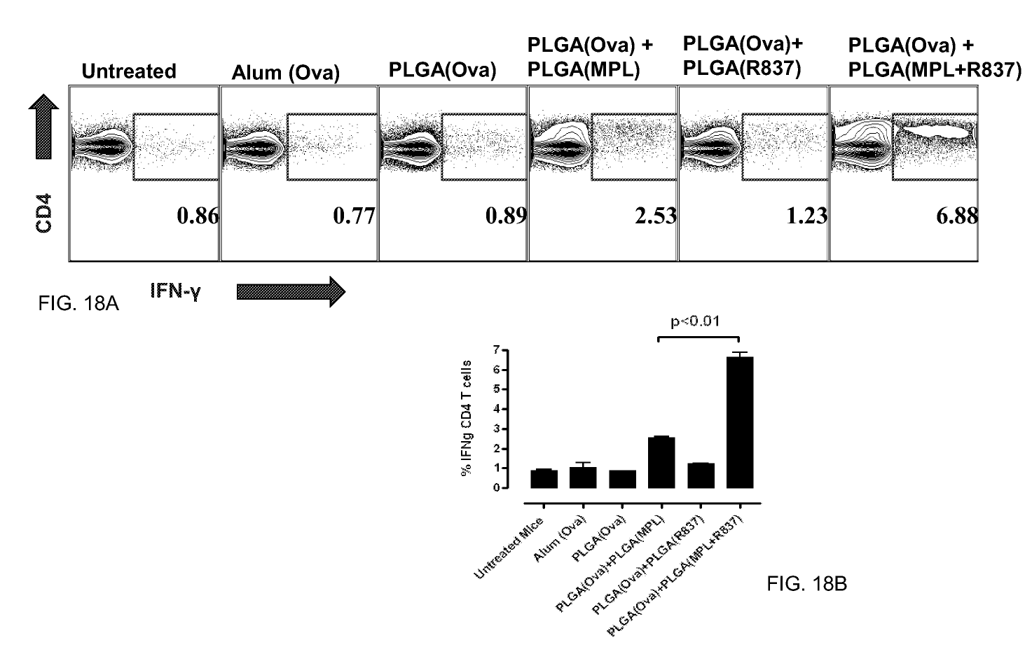

To determine whether delivery of PLGA nanoparticles containing MPL, R837 or both results in a synergistic CD4⁺ memory T cell response, C57BL6 mice were immunized with 10 μg of ovalbumin protein encapsulated in PLGA nanoparticles. MPL alone, R837 alone or a combination of MPL and R837 encapsulated in PLGA nanoparticles was co-delivered with ovalbumin encapsulated PLGA in nanoparticles to test for synergistic enhancement of memory CD4⁺ T cell responses. Mice were euthanized at 8 weeks post boost immunization by CO₂ asphyxiation. Cells were isolated from the inguinal lymph nodes by collagenase treatment for 45 minutes at 37° C. Cells (1×10⁶) were cultured in a 200 μl volume with 100 μg/ml of ovalbumin protein in 96 well plates for 4 days. Cells were transferred to anti-CD3 (10 μg/ml) and anti-CD28 (2 μg/ml) coated flat bottomed 96 well plates for 6 hours in the presence of Golgi plug (1 μg/ml) and Golgi stop (1 μg/ml). Cells were stained for CD4⁺ T cells and intracellular IFN-γ using established protocols. FIGS. 18A and 18B show the frequencies of CD4⁺ T cells that stained positive for IFN-γ cytokine, which indicates a potent CD4⁺ T cell response. The results demonstrate that combined delivery of MPL and R837 leads to a synergistic increase in the frequency of IFN-γ producing CD4⁺ T cells compared to immunization with MPL or R837 alone (with a sub-optimal dose of ovalbumin antigen).

### Example 8

### Co-Delivery of TLR Ligand-Containing Nanoparticles with Antigen-Containing Nanoparticles Mediates Synergistic Enhancement of Antibody Titers in Vivo

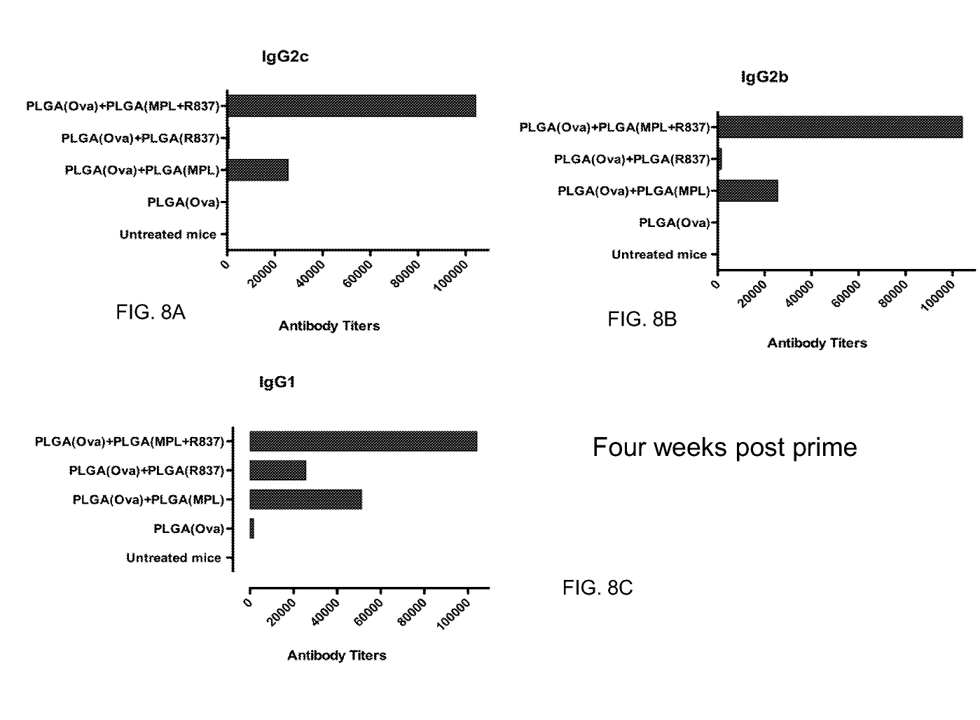

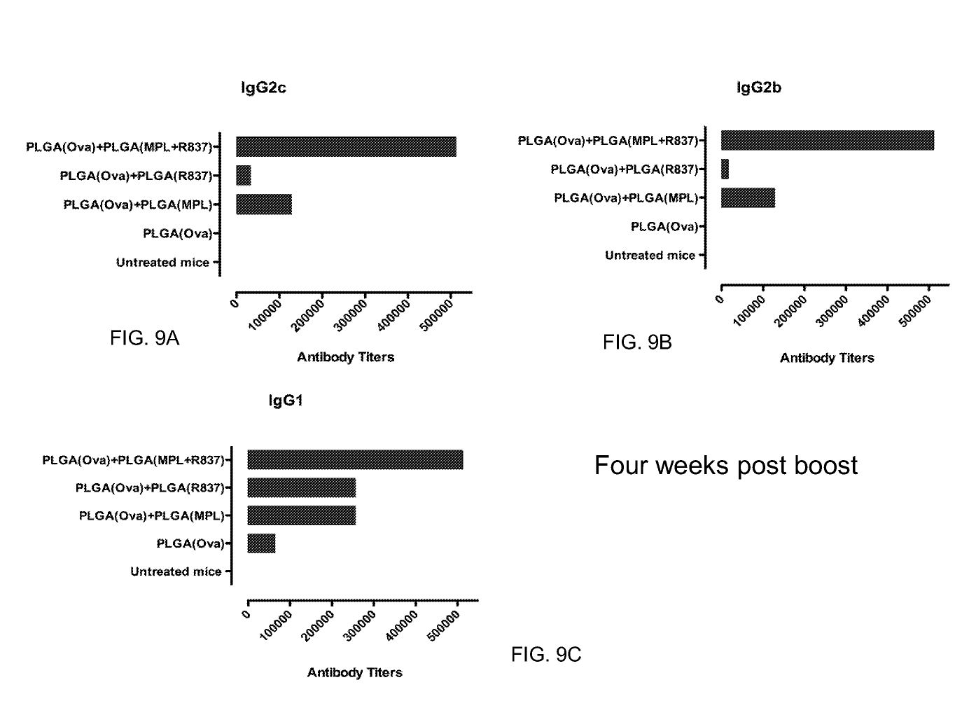

To determine whether delivery of nanoparticles containing both MPL and R837 resulted in a synergistic antibody response in vivo, 6-12 week old C57BL6 mice were immunized with 10 μg of soluble ovalbumin or ovalbumin encapsulated in PLGA nanoparticles. Some mice were also administered nanoparticles containing MPL, nanoparticles containing R837 or nanoparticles containing both MPL and R837. The doses of MPL and R837 were approximately 36 and 60 μg, respectively. Mice were bled via the lateral tail vein at regular intervals (days 15 and 28) after primary and secondary immunizations and serum was isolated for analysis of antibody responses by ELISA. Shown in FIGS. 8A-8C are the antibody isotype profiles at day 28 post primary immunization. The results demonstrate that co-delivery of nanoparticles containing both TLR ligands (MPL+R837) mediates synergistic enhancement of IgG_{2b}, IgG_{2c} and IgG₁ antibody titers compared to co-delivery of each individual TLR ligand. The results shown in FIGS. 9A-9C demonstrate synergistic enhancement of antibody titers in the same group of mice analyzed at day 28 post boost. In summary, delivery of co-encapsulated TLR ligands leads to synergistic amplification of antibody responses against a model protein antigen in vivo.

### Example 9

### Co-Delivery of TLR Ligand-Containing Nanoparticles with Anthrax Protective Antigen (AP)-Containing Nanoparticles Mediates Synergistic Enhancement of Antibody Titers in Vivo

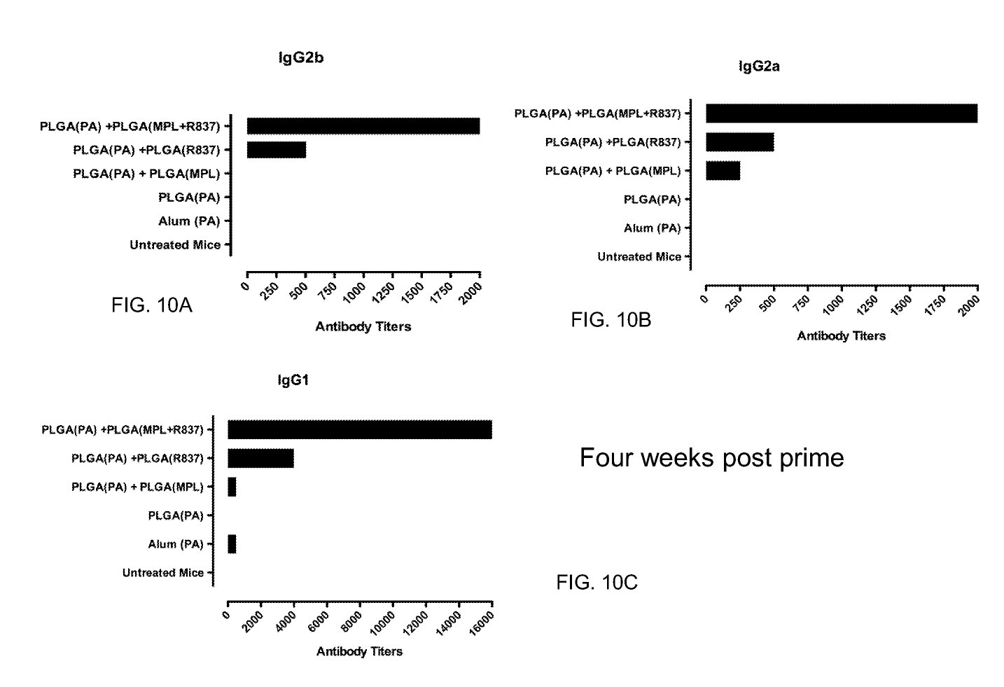

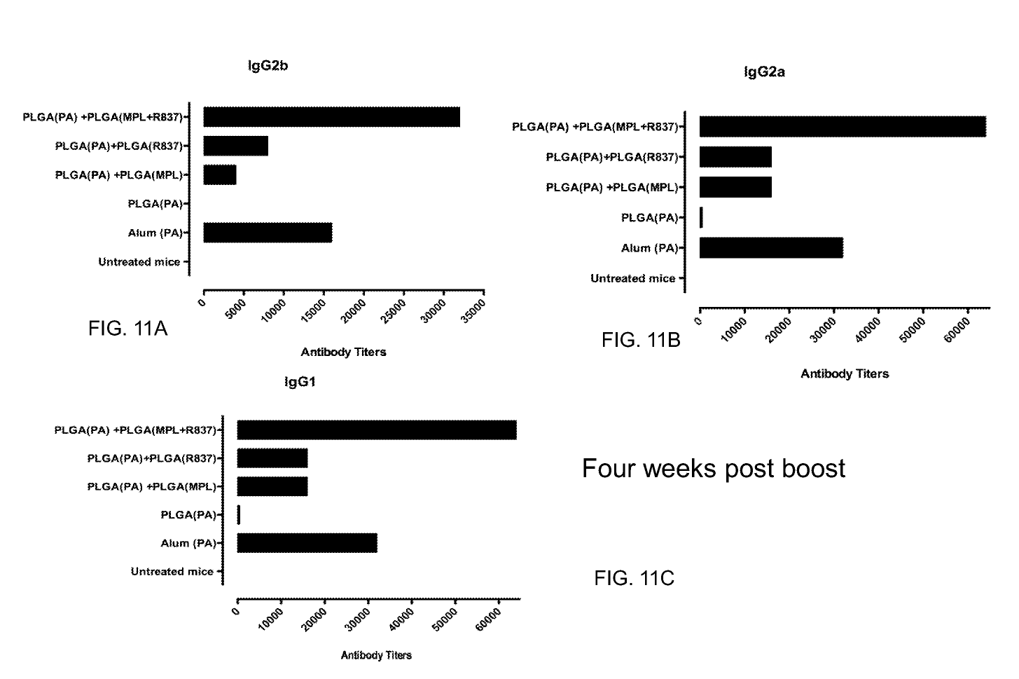

To further evaluate the synergistic antibody response following delivery of nanoparticles containing TLR ligands MPL and R837, 6-12 week old Balb/c mice were immunized with 10 μg of recombinant soluble PA or recombinant PA encapsulated in PLGA nanoparticles. Some mice were also administered nanoparticles containing MPL, nanoparticles containing R837, or nanoparticles containing both MPL and R837. The doses of MPL and R837 were approximately 36 and 60 μg, respectively. Mice were bled via the lateral tail vein at regular intervals (days 15 and 28) after primary and secondary immunizations and serum was isolated for analysis of antibody responses by ELISA. Shown in FIGS. 10A-10C are the antibody isotype profiles at day 28 post primary immunization. The results demonstrate that co-delivery of nanoparticles containing both TLR ligands (MPL+R837) mediates synergistic enhancement of IgG_{2b}, IgG₂ₐ and IgG₁ antibody titers compared to co-delivery of each individual TLR ligand. The results shown in FIGS. 11A-11C demonstrate synergistic enhancement of antibody titers in the same group of mice at day 28 post boost. In summary, delivery of co-encapsulated TLR ligands leads to synergistic amplification of antibody responses against anthrax-specific PA protein in vivo.

### Example 10

### Co-Delivery of TLR Ligand-Containing Nanoparticles with Anthrax Protective Antigen (AP)-Containing Nanoparticles Mediates Synergistic Enhancement of High Affinity Antibodies

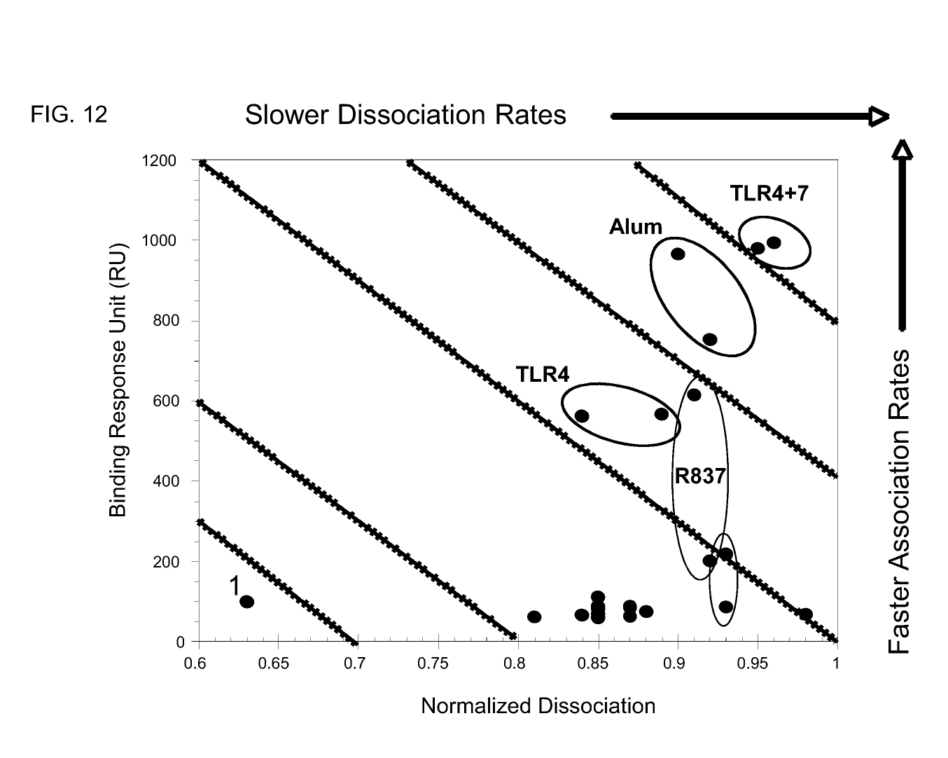

Serum samples from PA immunized mice were tested for serum antibody binding affinity (antigen/antibody association kinetics) as well as stability of serum antibody binding to PA antigen using the BIACORE™ protein characterization system (GE Healthcare, Milwaukee). As shown in FIG. 12, delivery of TLR ligands MPL and R837 encapsulated in the same nanoparticles leads to production of higher affinity antibodies compared with delivery of a single TLR ligand. These high affinity antibodies also displayed rapid association kinetics (high Kₐ) and slow dissociation kinetics (low K_{d}), which indicates stable binding at equilibrium over the measured interval of time. In summary, combined delivery of TLR ligands not only leads to high antibody titers (FIGS. 10 and 11), but also results in production of high affinity antibodies (FIG. 12).

### Example 11

### Co-Delivery of TLR Ligand-Containing Nanoparticles with H5HA-Containing Nanoparticles Mediates Synergistic Enhancement of Anti-HA Antibody Responses and Produces Antibodies with High Avidity

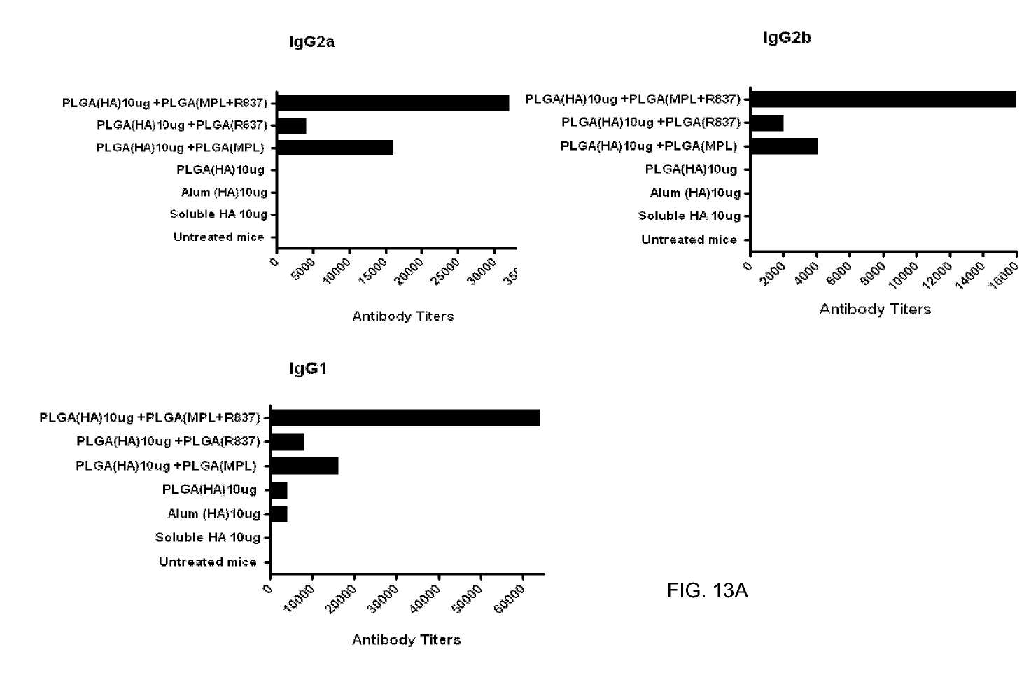

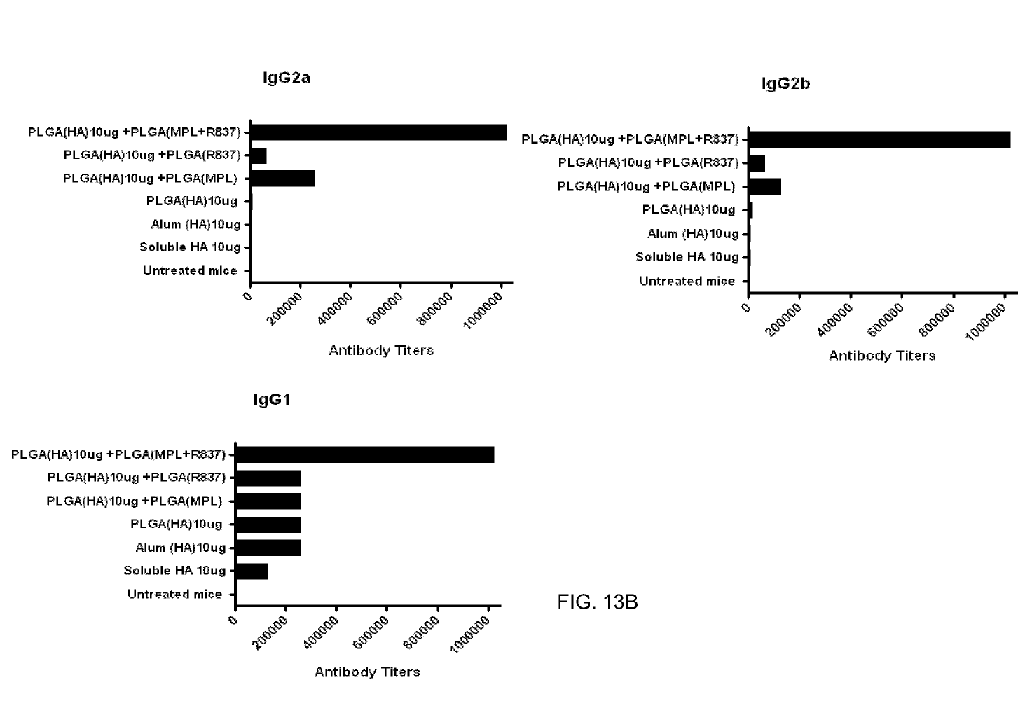

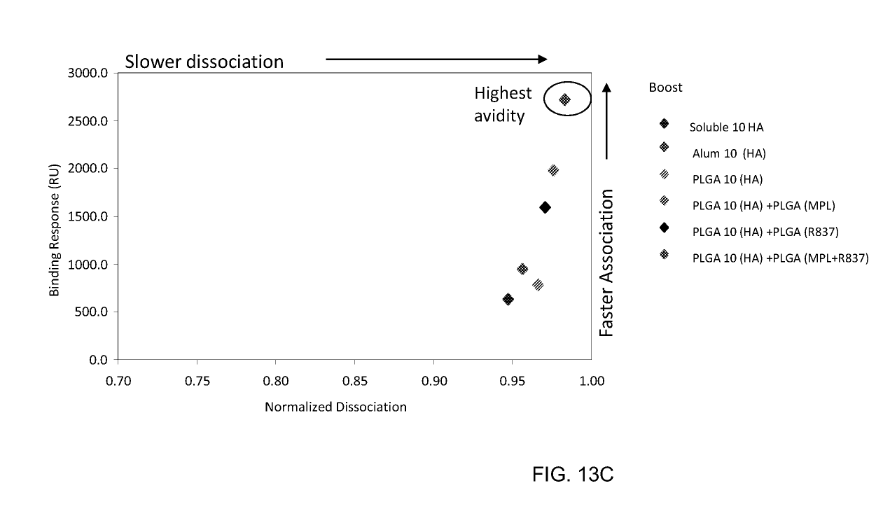

To further evaluate the synergistic effect of MPL and R837 delivered in combination with a different antigen, 6-12 week old Balb/c mice were immunized with 10 μg of recombinant soluble avian influenza H5HA or H5HA encapsulated in PLGA nanoparticles. Nanoparticles containing MPL, nanoparticles containing R837 or nanoparticles containing both MPL and R837 were co-delivered with PLGA-encapsulated H5HA. The doses of MPL and R837 were approximately 36 and 60 μg, respectively. Mice were bled via the lateral tail vein at regular intervals (days 15 and 28) after primary and secondary immunizations, and serum was isolated for analysis of antibody responses by ELISA. As shown in FIG. 13A, co-delivery of nanoparticles containing both TLR ligands (MPL+R837) mediated synergistic enhancement of IgG₂ₐ, IgG_{2b} and IgG₁ antibody titers compared to co-delivery of individual TLR ligands after primary immunization. The results shown in FIG. 13B demonstrate synergistic enhancement of antibody titers in the same group of mice analyzed at day 28 post boost. Thus, combined delivery of TLR ligands leads to synergistic amplification of antibody responses against avian influenza specific HA protein in vivo.

Serum samples from the H5HA immunized mice were tested for binding affinity (antigen/antibody association kinetics) as well as stability of serum antibody binding to H5HA antigen using the BIACORE™ antigen/antibody binding characterization system (GE Healthcare, Milwaukee, USA). Combined delivery of TLR ligands encapsulated in nanoparticles compared to single TLR ligand adjuvant delivery with encapsulated proteins led to production of high avidity antibodies (FIG. 13C). These antibodies also displayed rapid association kinetics (high Kₐ) as well as slow dissociation kinetics (low K_{d}), which indicates stable binding at equilibrium over the measured interval of time. Thus, combined delivery of TLR ligands not only leads to high antibody titers, but also ensures high affinity of these antibodies.

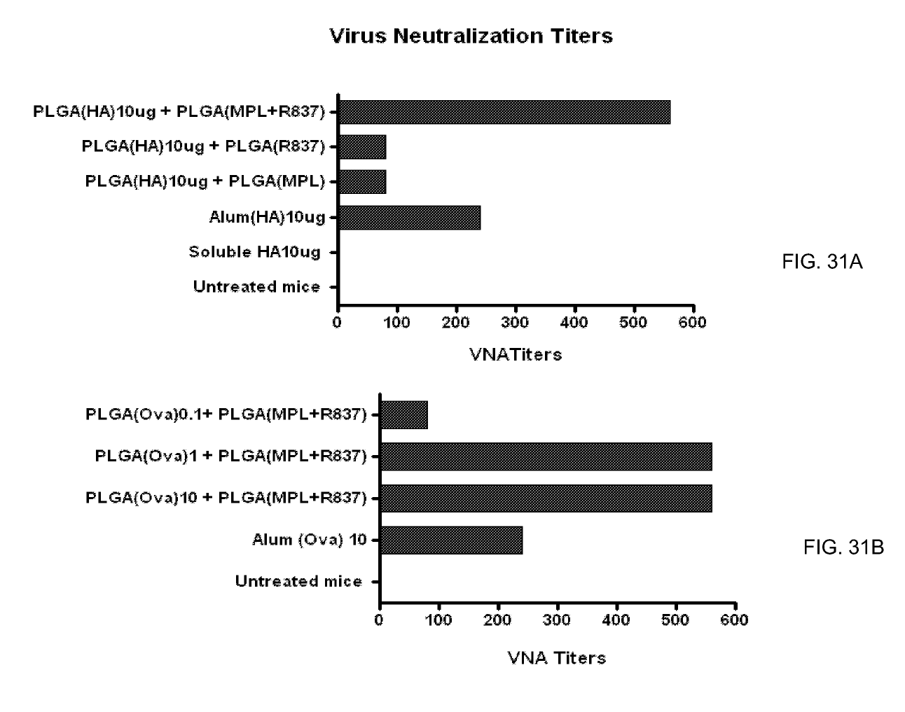

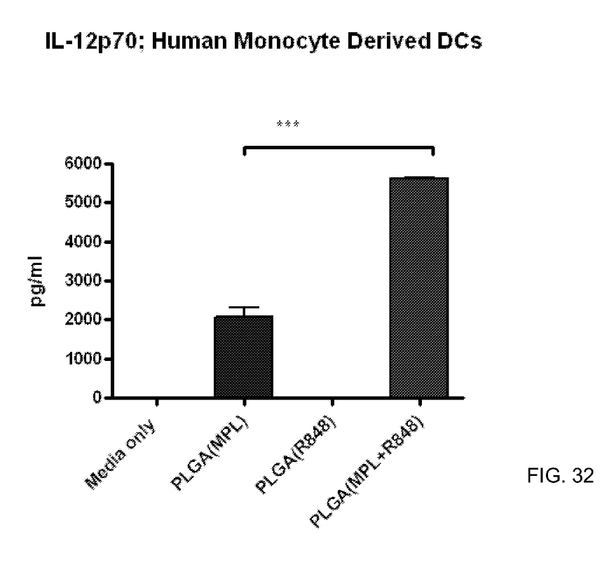

To evaluate the effect of TLR ligand-containing nanoparticles on neutralizing antibody titers, serum samples from H5HA immunized mice were tested for their ability to neutralize H5HA-expressing influenza A virus (A/PR/8/34) in cell culture. MDCK cells were cultured with virus-treated serum samples for 48 hours. Cell culture supernatants were subjected to an established hemagglutinin inhibition (HAI) assay to test for the presence of virus particles. As shown in FIG. 31A, the combination of TLR ligands MPL and R837 mediates a synergistic increase in virus neutralization titers compared to treatment with a single TLR ligand. In addition, immunization with the combination of MPL and R837 results in a significant enhancement in virus neutralization titers compared with the clinically approved Alum adjuvant. FIG. 31B shows that a 10-fold lower antigen dose in nanoparticles injected with the combination of TLR ligands elicits greater responses than the clinically approved Alum adjuvant.

### Example 12

### Combined Delivery of TLR Ligands MPL and R837 Leads to Polyclonal Stimulation of Naïve B Cells in Vitro and Synergistic Antibody Production Dependent on MyD88 and TRIF

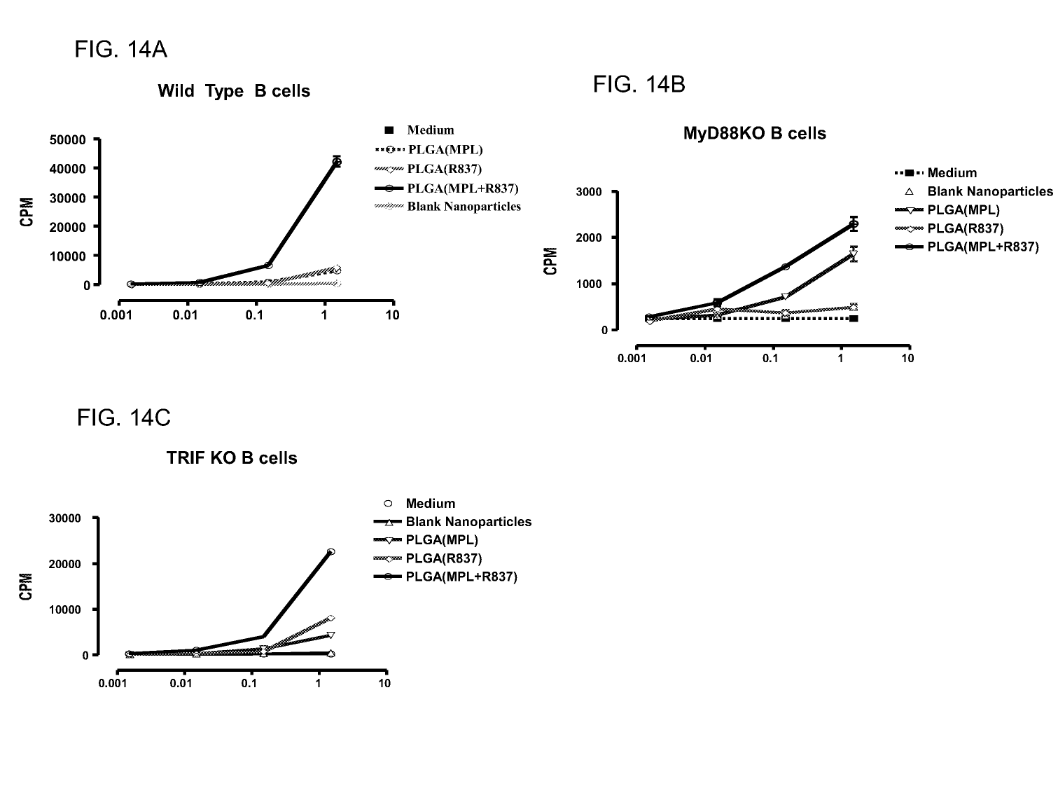

Naïve B cells were isolated from C57BL6 mice using anti-CD19 magnetic beads (Miltenyi Biotec, Auburn, Calif.). Wild-type naïve B cells, MyD88 knockout naïve B cells and TRIF (TIR-domain-containing adapter-inducing interferon-β) knockout naïve B cells were cultured at 200,000 cells per well in a round bottom 96-well plate with either medium alone, blank nanoparticles, nanoparticles containing MPL, nanoparticles containing R837 or nanoparticles containing both MPL and R837. MPL doses were titrated at 1.5, 0.15, 0.015, 0.0015 μg/ml and R837 doses were titrated at 2, 0.2, 0.02, 0.002 μg/ml. ³H-thymidine was added after 48 hours of culture for an additional 12 hours and cells were harvested using a Tomtec (Hamden, Conn.) cell harvester onto a filter mat and radioactivity read using a beta counter. The results, shown in FIGS. 14A-14C, are reported as proliferation of B cells as indicated by counts per minute (CPM) of ³H-thymidine incorporated in proliferating cells. The results indicate that the proliferation of naïve B cells in response to a combination of TLR ligands is heavily dependent on MyD88 and partially dependent on TRIF signaling pathways, which are currently known to assist TLR ligand signaling.

To further evaluate the role of MyD88 and TRIF in TLR signaling, 8-12 week old C57BL6 mice were immunized with 10 μg of soluble ovalbumin (Alum)(Ova)) or PLGA-encapsulated ovalbumin (WT) in combination with PLGA nanoparticles containing both MPL and R837 (FIGS. 15A-15C). Antibody responses in wild type mice were compared with MyD88-deficient (MyD88KO) and TRIF-deficient (TRIFKO) mice. In addition, responses were compared with mice depleted of DCs (CD11cDTR), macrophages (Clodronate Liposome KO) or CD4+ T (Anti-CD4) cells to test the effect of each of these cells types on TLR signaling. CD11c diphtheria toxin receptor (DTR) mice (Jung et al., Immunity 17:211-220, 2002; van Rijt et al., J. Exp. Med. 201(6):981-991, 2005) were injected with diphtheria toxin (DT) (650 ng) 24 hours before immunization with ovalbumin, followed by a 100 ng DT dose at day 3 post immunization. Macrophages were depleted using clodronate liposomes in the draining lymph nodes at the site of injection 5 days prior to immunization. For depletion of CD4⁺ T helper cells, recombinant anti-CD4 antibody GK1.5 (Dialynas et al., Immunol Rev. 74:29-56, 1983) was administered to mice intraperitoneally at a dose of 250 μg on days-4, -2 and +3 relative to Ova immunization.

Mice were bled via the lateral tail vein at regular intervals (days 15 and 28) after primary and secondary immunizations. Serum was isolated for analysis of antibody responses by ELISA. As shown in FIGS. 15A-15C, antibody titers (analyzed at day 28 after primary immunization) were reduced most significantly in MyD88-deficient, TRIF-deficient, DC-depleted and CD4+ T helper cell-depleted mice. In contrast, depletion of macrophages led to a striking increase in antibody titers. These data demonstrate that the combination of TLR Ligands MPL and R837 mediates synergistic antibody responses that are dependent on MyD88 and TRIF adaptor proteins, which are important for TLR signaling. The results further indicate that the synergistic antibody response is also dependent on CD11c⁺ DCs and CD4⁺ T helper cells, but not on macrophages.

### Example 13

### Synergistic Increases in Antibody Responses are Dependent on the Presence of Dendritic Cells (DCs)

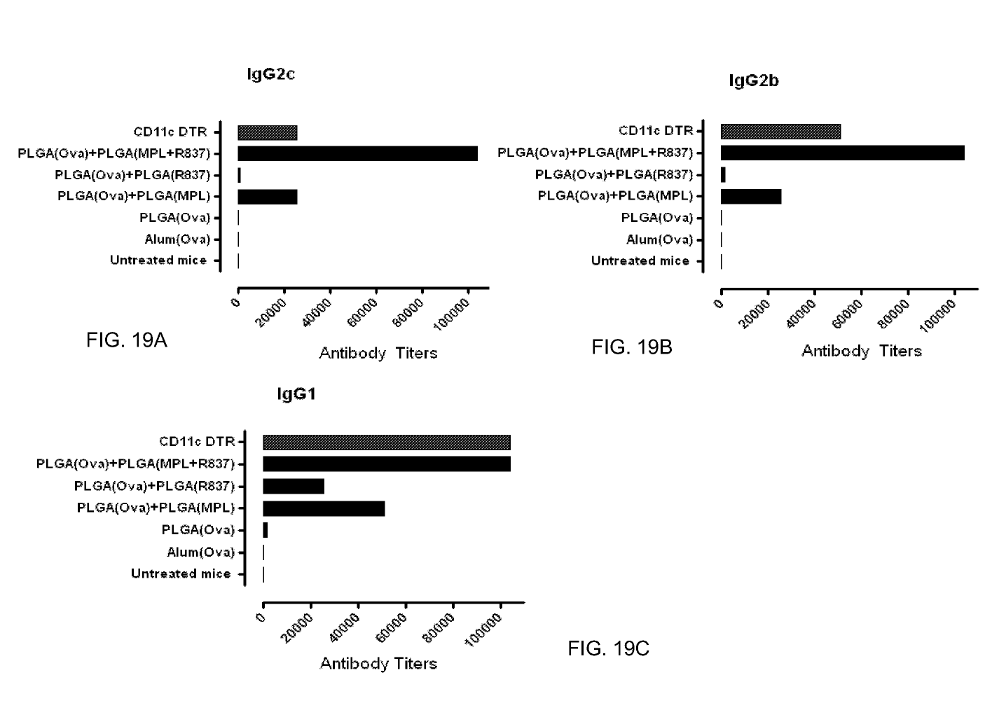

To determine whether dendritic cells (DCs) are important for the observed synergistic enhancement of antibody responses following administration of TLR ligands, 6-12 week old C57BL6 mice and CD11c-DTR mice were immunized with 10 μg of ovalbumin protein encapsulated in PLGA nanoparticles. PLGA nanoparticles containing both MPL and R837 were co-delivered with ovalbumin encapsulated PLGA nanoparticles to compare antibody responses in DC-sufficient and DC-depleted mice. CD11c-DTR mice carry a diphtheria toxin receptor (DTR) driven by the CD11c promoter. This ensures that the diphtheria toxin receptor is selectively expressed in CD11c⁺ DCs. As a result, injection of diphtheria toxin (DT) (600 ng/mouse) results in transient depletion of DCs at the time of immunization. Mice were bled via the lateral tail vein at regular intervals (days 15 and 28) after primary and secondary immunizations and serum was isolated for analysis of antibody responses. Shown in FIGS. 19A-19C is the antibody isotype profiling by ELISA at day 28 post primary immunization. The results demonstrate that depletion of DCs at the time of immunization results in a significant decrease in IgG2c and IgG2b (Th1 polarized) antibody titers, whereas IgG1 antibody titers were not dependent on the presence of DCs. In summary, the presence of DCs at the time of immunization with antigens and TLR ligands in nanoparticles can modulate the Th1 versus Th2 profile of antibody responses in mice.

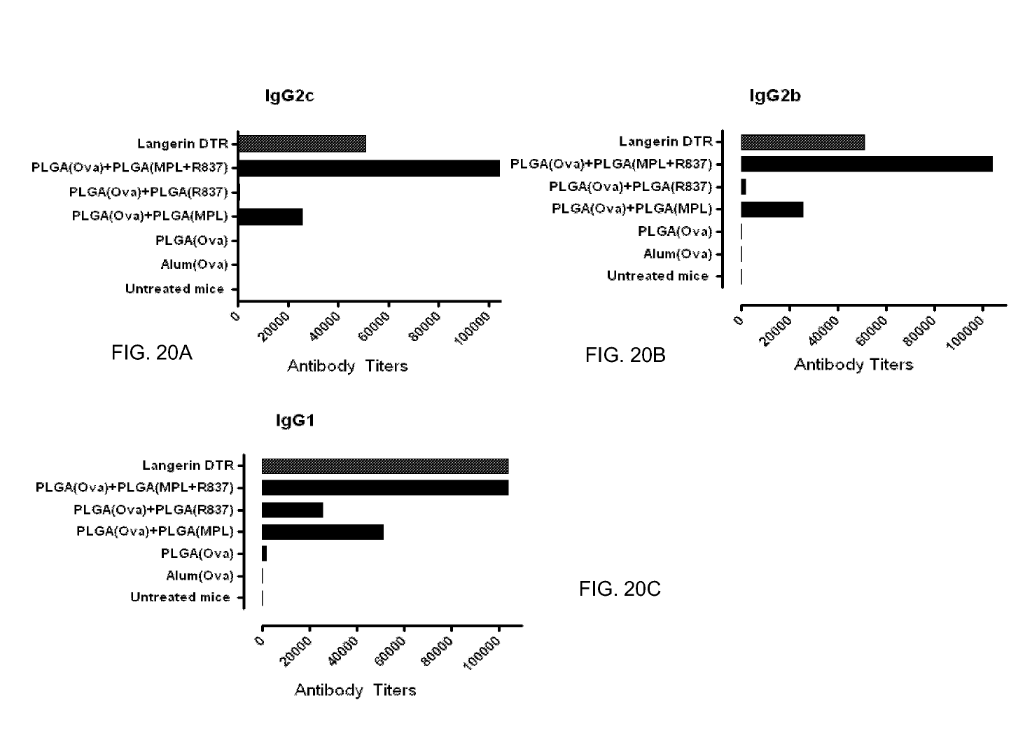

To specifically evaluate the role of Langerhans cells, a type of DC, 6-12 week old C57BL6 mice and Langerin-DTR mice were immunized with 10 μg of ovalbumin protein encapsulated in PLGA nanoparticles. PLGA nanoparticles containing the combination of MPL and R837 were co-delivered with ovalbumin encapsulated in PLGA to compare antibody responses in DC-sufficient and DC-depleted mice. Langerin-DTR mice carry a diphtheria toxin receptor driven by the Langerin promoter. This ensures that the diphtheria toxin receptor is selectively expressed in Langerin⁺ DCs (Langerhans cells). As a result, upon injection of DT (600 ng/mouse), transient depletion of Langerhans cells occurs at the time of immunization. Mice were bled via the lateral tail vein at regular intervals (days 15 and 28) after primary and secondary immunizations and serum was isolated for analysis of antibody responses by ELISA. Shown in FIGS. 20A-20C is the antibody isotype profiling at day 28 post primary immunization. The results show that depletion of Langerhans cells at the time of immunization mediated a significant decrease in IgG2c and IgG2b (Th1 polarized) antibody titers, whereas IgG1 antibody titers are not dependent on the presence of Langerhans cells. In summary, the presence of Langerhans cells at the time of immunization with antigens and TLR ligands in nanoparticles can modulate the Th1 versus Th2 profile of antibody responses in mice.

### Example 14

### Synergistic Increases in Antibody Responses with TLR-Ligand Containing Nanoparticles are Dependent on Presence of Pro-Inflammatory Cytokines

To evaluate the role of pro-inflammatory cytokines on the synergistic enhancement of antibody responses following delivery of nanoparticles containing TLR ligands, 6-12 week old C57BL6 mice, IL-6^{-/-} mice, B6129 mice and interferon receptor-α receptor knockout (IFNαR^{-/-}) mice were immunized with 10 μg of ovalbumin protein encapsulated in PLGA nanoparticles. Combination (MPL and R837) nanoparticles were co-delivered with ovalbumin encapsulated in PLGA nanoparticles to compare antibody responses in IL-6 cytokine-sufficient and -deficient mice as well as type-1 interferon receptor-sufficient and -deficient mice. IL-6^{-/-} mice are deficient in IL-6 cytokine secretion and the (IFNαR^{-/-}) mice are deficient in type-1 interferon receptors on all cell types. As a result, these mice lack the ability to secrete IL-6 or respond to type 1 interferon during the course of the immune response.

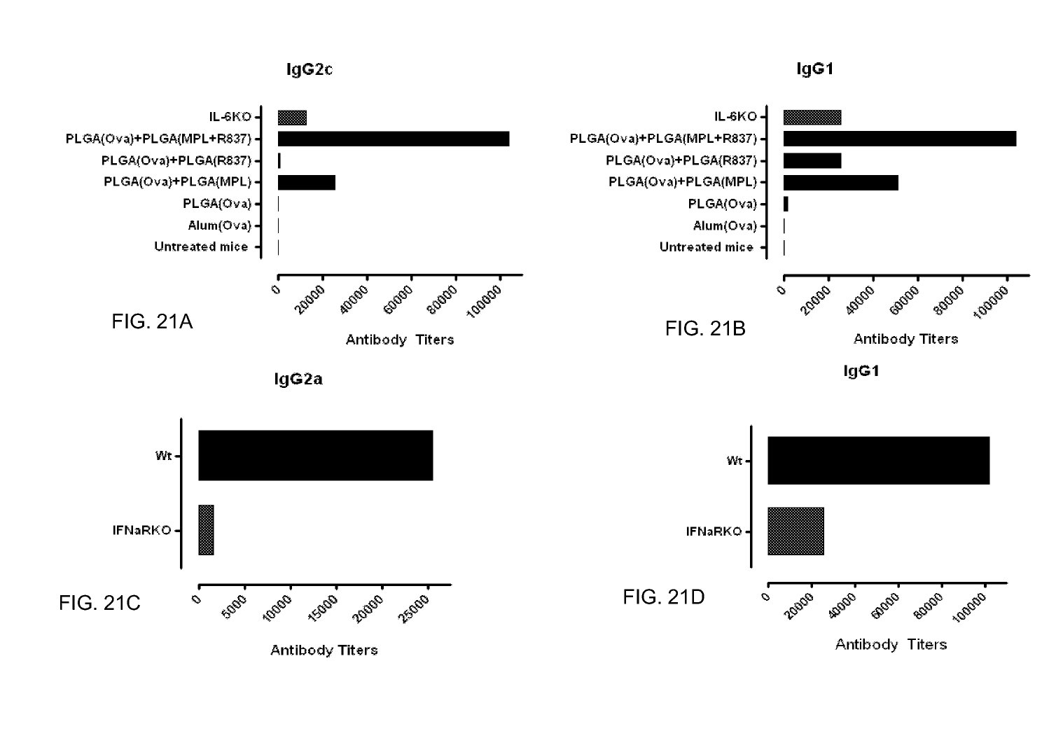

Mice were bled via the lateral tail vein at regular intervals (days 15 and 28) after primary and secondary immunizations and serum was isolated for analysis of antibody responses by ELISA. Shown in FIGS. 21A-21D is the antibody isotype profiling at day 28 post primary immunization. The results demonstrate that the absence of IL-6 or type I interferon receptor leads to a significant decrease in IgG2c (Th1 response in C57BL6 mice) and IgG2a (Th1 response in B6.129 mice) antibody titers as well as IgG1 (Th2 response) antibody titers. Thus, the presence of IL-6 and the ability to respond to type 1 interferons during the course of the immune response with antigens and TLR ligands in nanoparticles are critical for the efficient induction of antigen-specific antibodies in mice.

### Example 15

### Synergistic Increases in Antibody Responses are Dependent on the Presence of CD4⁺ T Cells

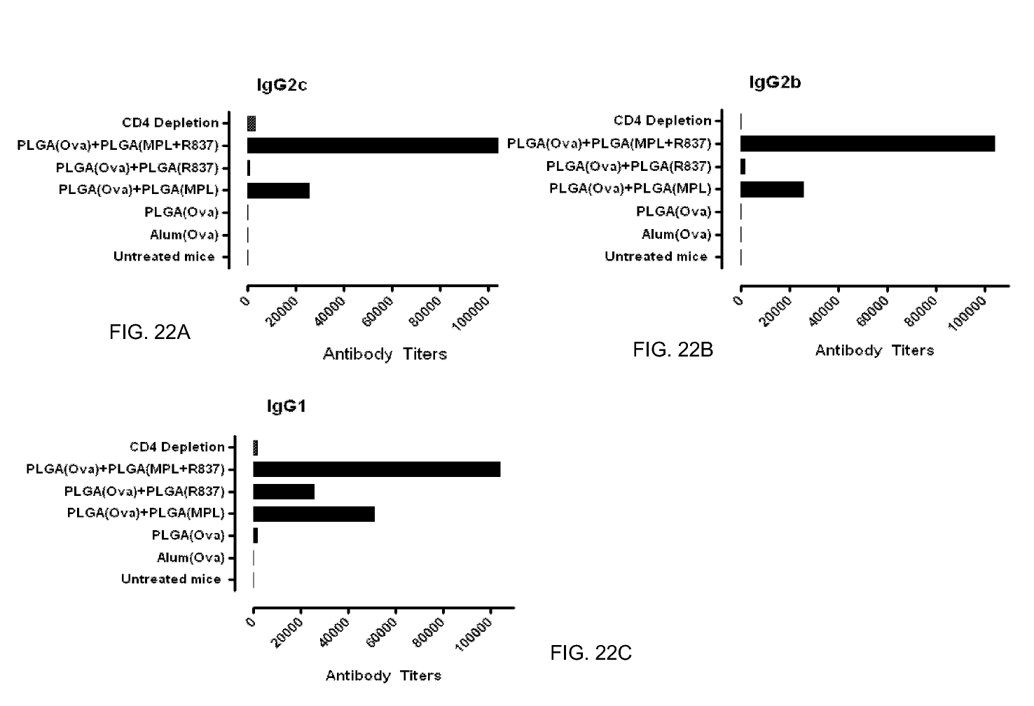

To evaluate the role of CD4⁺ T cells in the observed synergistic antibody responses following immunization with TLR ligands, 6-12 week old C57BL6 mice were injected with GK1.5 anti-CD4 antibody at 250 μg/mouse intraperitoneally on days-3 and -1 before immunization and day +3 after primary immunization. Injection of GK1.5 anti-CD4 antibody depleted all CD4⁺ cells with greater than 98% efficiency. As a result, these mice lack any help from antigen primed CD4⁺ cells during the course of the immune response. Mice were bled via the lateral tail vein at regular intervals (days 15 and 28) after primary and secondary immunizations and serum was isolated for analysis of antibody responses by ELISA. Shown in FIGS. 22A-22C is the antibody isotype profiling at day 28 post primary immunization. The results show that the absence of CD4⁺ T cells during the course of the primary immune response leads to a significant decrease in all antibody isotypes (IgG2c, IgG2b and IgG1). Therefore, the presence of CD4⁺ T cell help is critical for the synergistic increase of antibody titers upon treatment with MPL and R837 as adjuvants.

### Example 16

### Synergistic Increase in Antibody Response Due to TLR Ligands is Dependent on TLR Signaling in B Cells

To determine whether direct TLR signaling in B cells is required for synergy in vivo, B cells from wild-type, MyD88^{-/-} or TRIF^{-/-} mice were transferred into μMT mice, which lack mature B cells. B cells were purified from spleens and lymph nodes of C57BL6, MyD88^{-/-} and TRIF^{-/-} mice using positive selection with anti-CD19⁺ magnetic beads (Miltenyi Biotec, Auburn, Calif.). Naïve B cells were greater than 95% pure as evaluated by flow cytometry. Naïve B cells (40×10⁶) from the above-mentioned mice strains were transferred into 3 μMT mice per group and immunized with ovalbumin antigen and MPL+R837 in nanoparticles five days after B cell reconstitution. This experimental design was used to create a mouse model in which only the B cells were sufficient or deficient in signaling via MyD88 or TRIF. All other cell types were capable of efficiently responding to MPL and R837 stimulation.

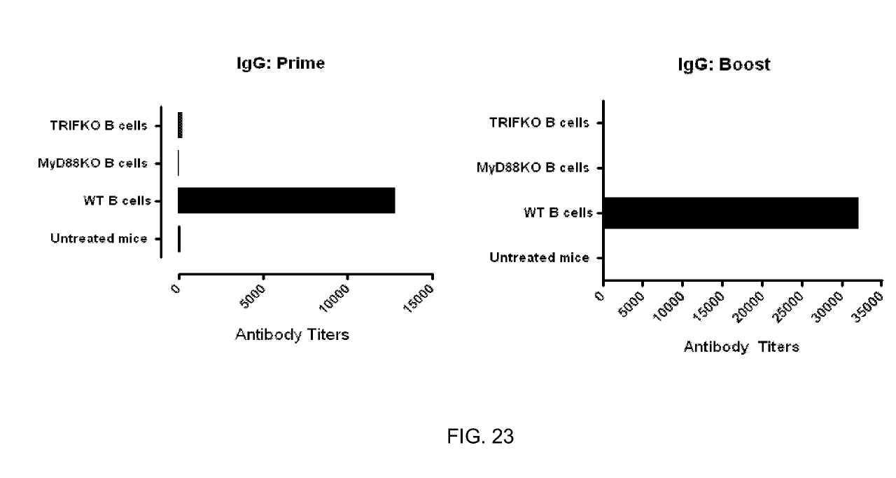

Mice were bled via the lateral tail vein at regular intervals (days 15 and 28) after primary and secondary immunizations and serum was isolated for analysis of antibody responses by ELISA. Shown in FIG. 23 are the antibody titers analyzed at day 28 post primary and secondary immunization. The results show that the absence of MyD88 or TRIF on B cells during the course of the immune response resulted in a significant decrease in total IgG (IgG1+IgG2+IgG3) antibody titers. In summary, the presence of TLRs on B cells is critical for the synergistic increase of antibody titers upon treatment with MPL+R837 as adjuvants with protein antigens.

To determine whether TLR4 and TLR7 must be expressed on the same B cell to allow for the synergistic increase in antibody response due to MPL+R837, B cells from wild-type, TLR-deficient or TLR7-deficient mice were transferred into μMT mice, which lack mature B cells. B cells were purified from spleens and lymph nodes of C57BL6, TLR4^{-/-} and TLR7^{-/-} mice using positive selection with anti-CD19⁺ magnetic beads (Miltenyi Biotec, Auburn, Calif.). Naïve B cells were greater than 95% pure as evaluated by flow cytometry. Groups of 3 μMT mice were reconstituted via intravenous injections with 40×10⁶ naïve B cells from the above mentioned mice strains and immunized 5 days later with ovalbumin antigen and MPL+R837 in nanoparticles. One group of 3 μMT mice was reconstituted with 20×10⁶ TLR4^{-/-} B cells and 20×10⁶ TLR7^{-/-} B cells to create a mouse model in which half of the B cells are deficient in responding to MPL and the other half of the B cells are deficient in responding to R837. This experimental design was used to test if both TLR4 and TLR7 were needed on the same B cell for efficient induction of antibody responses. All the other cell types were capable of efficiently responding to MPL+R837 stimulation.

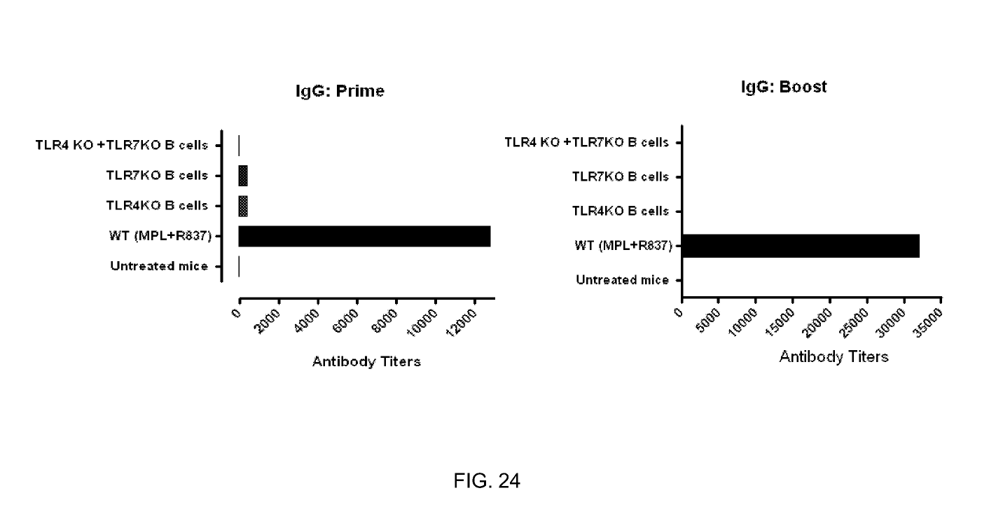

Mice were bled via the lateral tail vein at regular intervals (days 15 and 28) after primary and secondary immunizations and serum was isolated for analysis of antibody responses by ELISA. Shown in FIG. 24 is the antibody isotype profiling at day 28 post primary immunization. The results show that the absence of TLR4 and TLR7 on B cells during the course of the immune response resulted in a significant decrease in total IgG (IgG1+IgG2+IgG3) antibody titer. In addition, mice lacking both TLR4 and TLR7 on the same B cell had a significant reduction in total IgG antibody titers. In summary, the presence of TLRs on B cells, and more importantly the presence of both TLR4 and TLR7 on the same B cell, is critical for the synergistic increase of antibody titer following treatment with MPL+R837 as adjuvants with protein antigens.

### Example 17

### Co-Delivery of TLR Ligand-Containing and Antigen-Containing Nanoparticles Mediates Synergistic Increases in Persistence of Antigen Specific B Cells and the Number of Germinal Centers

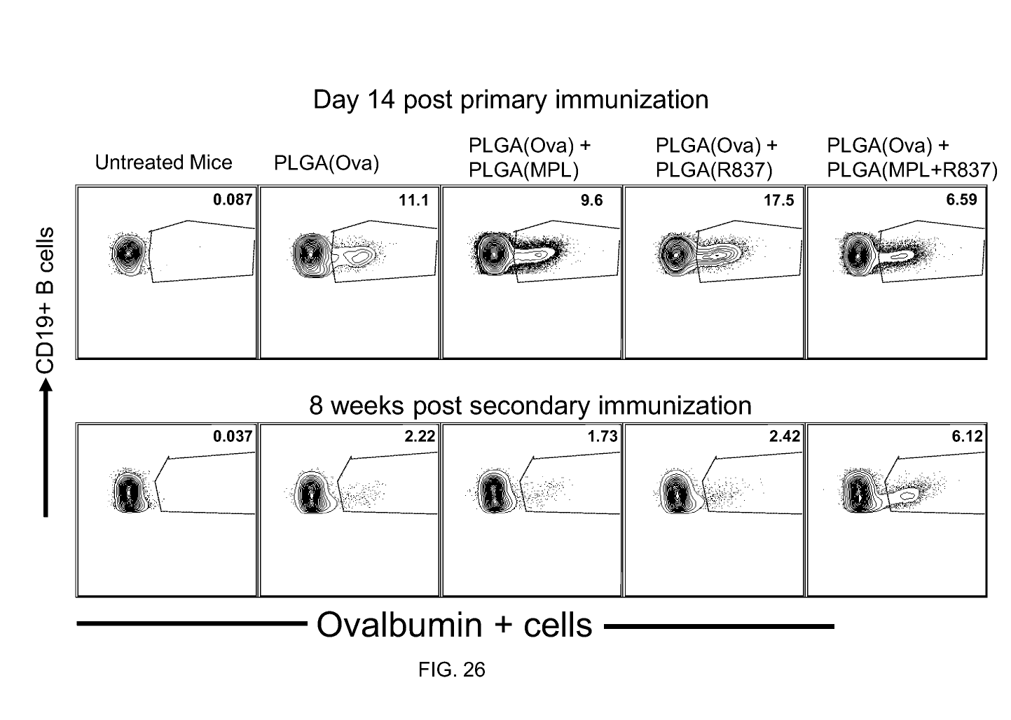

Antigen-specific B cell responses were evaluated with multi-color flow cytometry as follows. Lymph nodes were processed as described above in Example 3 by treatment with collagenase type IV for 45 minutes at 37° C. Isolated lymph node cells were fluorescently labeled as indicated in FIG. 25. CD19⁺ B cells were selected by gating and excluding all T cells and myeloid cells (TCRbeta⁺, CD11b⁺). All naïve B cells that were TCR⁻CD11b⁻CD19⁺IgD⁻ were excluded and class switched IgG (1+2+3) B cells were selected for further analysis. All TCR⁻CD11b⁻CD19⁺IgD⁻IgG⁺B cells were further classified as Ovalbumin⁺ antigen-specific cells or GL7⁺ germinal center cells or CD138⁺ plasma cells. As indicated in FIG. 26, there were no differences in the frequencies of ovalbumin-specific B cells at day 14 post primary immunization. In addition, immunization with MPL+R837 induced synergistic increases in the frequencies of antigen-specific B cells post prime and post boost immunization. These experiments suggest that the combination of MPL+R837 as adjuvants with protein antigens yields long lived memory B cell responses.

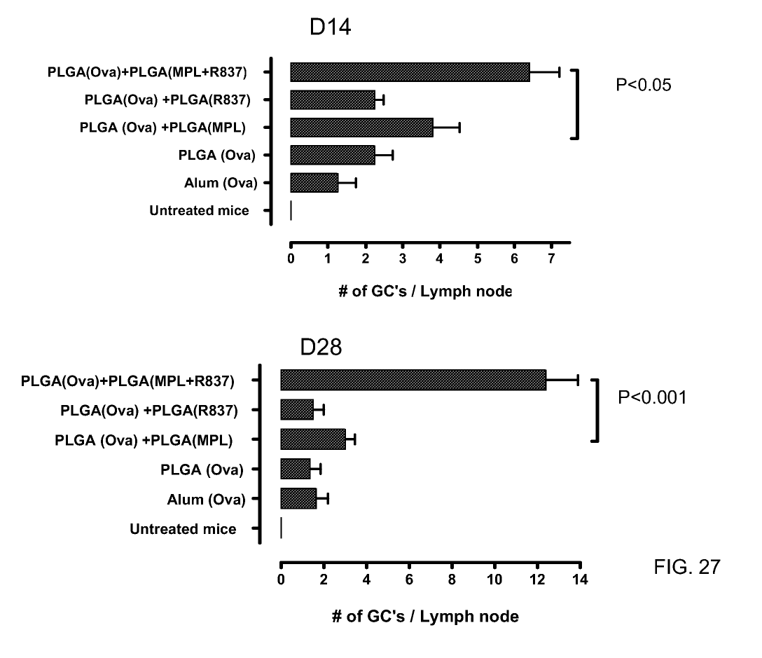

To evaluate the effect of co-delivery of TLR ligand-containing nanoparticles and antigen-containing nanoparticles on the number of germinal centers in draining lymph nodes, 6-8 week old C57BL6 mice were immunized with ovalbumin protein encapsulated in nanoparticles along with either MPL encapsulated nanoparticles, R837 encapsulated in nanoparticles or a combination of MPL+R837 encapsulated in nanoparticles. Draining inguinal lymph nodes were surgically excised on day 14 and day 28 post primary immunization and frozen in OCT medium with 2-methyl butane that was cooled with liquid nitrogen. Frozen lymph nodes were sectioned using Leica cryostat equipment at 5 μm thickness. Frozen lymph node sections were fixed in ice cold acetone for 10 minutes, air dried and stored at −80° C. until use for immunohistology staining Lymph node sections were stained with anti-mouse IgG (Alexa-488), anti-mouse GL-7 biotin antibody followed by Streptavidin Alexa-555 conjugate, and anti-mouse B220-Alexa647 conjugate. The results demonstrated that the number of germinal centers was synergistically increased following immunization with MPL+R837 combination nanoparticles (FIG. 27). These experiments suggest that the combination of MPL+R837 as adjuvants mediates efficient formation and synergistic increases in the number of germinal centers in the draining lymph nodes of mice that last for at least 6 weeks post primary immunization.

### Example 18

### Co-Delivery of MPL+R837 Encapsulated in Nanoparticles with Ovalbumin Encapsulated in Nanoparticles Results in a Synergistic Increase in the Number of Antibody Secreting Cells (ASCs)

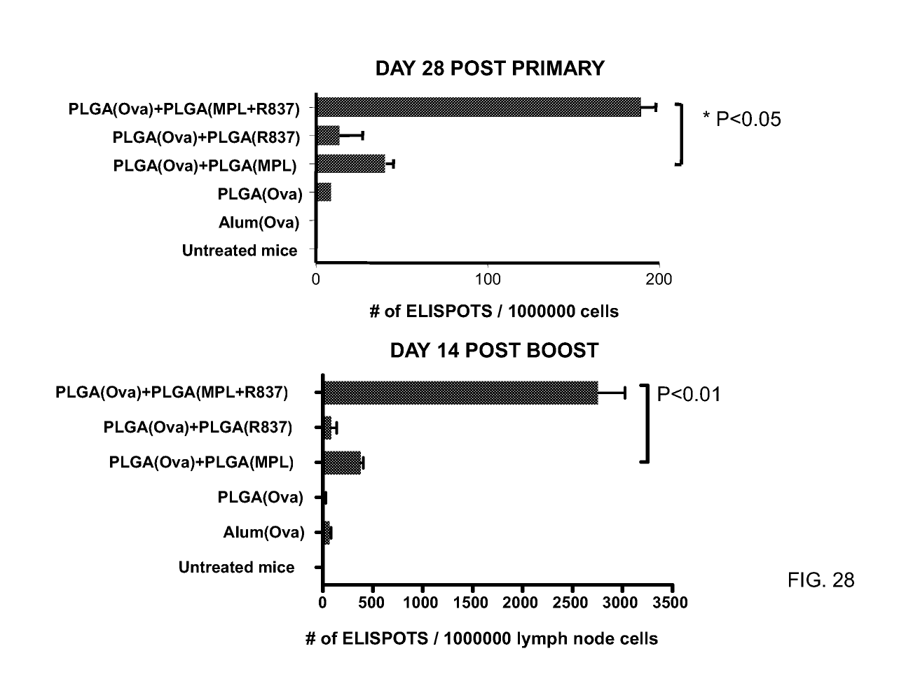

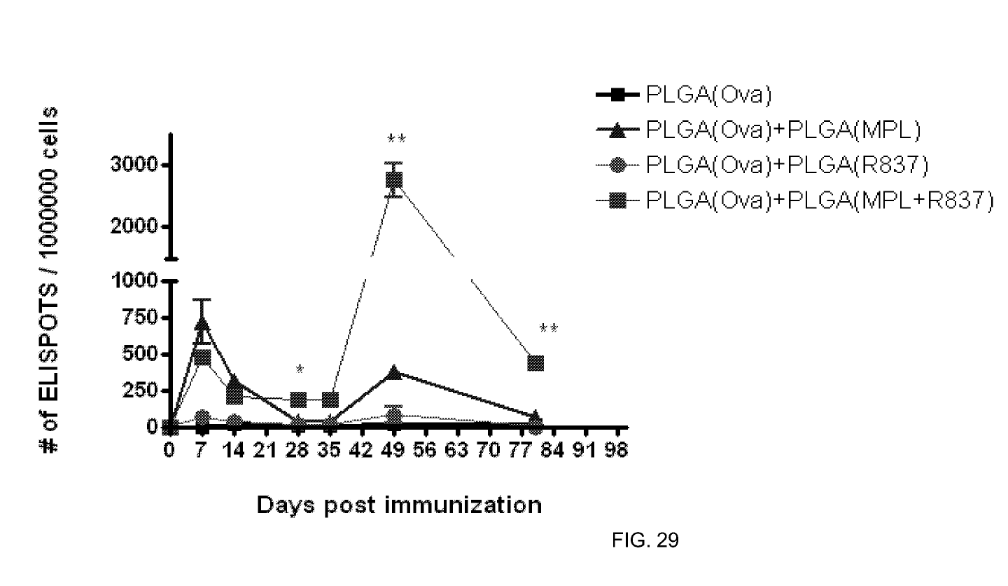

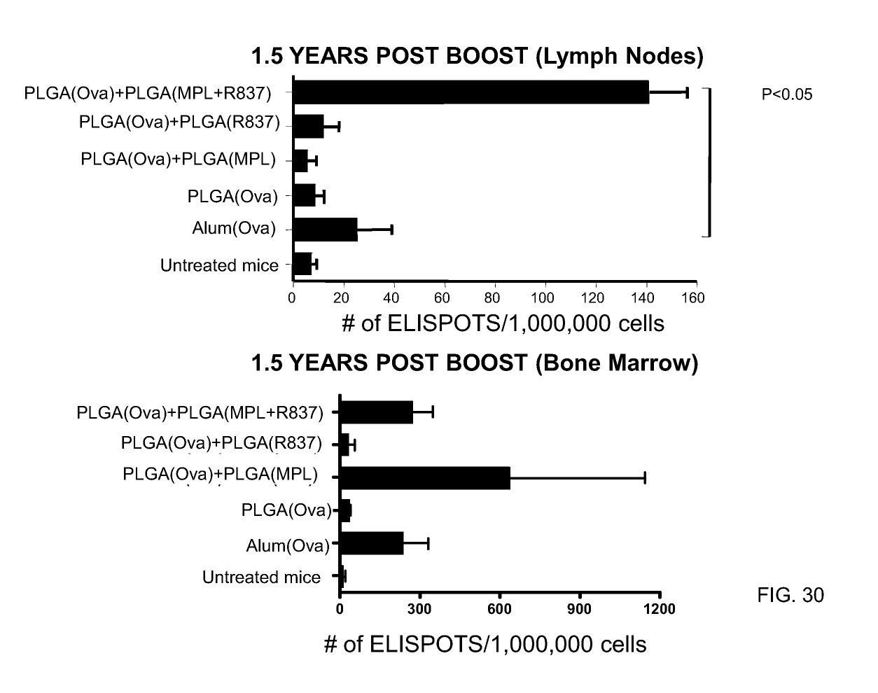

To evaluate the persistence of antibody forming plasmid cells in primary and memory responses, 6-8 week old C57BL6 mice were immunized with ovalbumin encapsulated in nanoparticles along with either MPL encapsulated in nanoparticles, R837 encapsulated in nanoparticles or a combination of MPL+R837 encapsulated in nanoparticles. Draining inguinal lymph nodes were surgically excised on days 7, 14 and 28 post primary immunization and days 14 and 56 post boost immunization. Lymph nodes were processed as described in Example 3 by treatment with collagenase type IV for 45 minutes at 37° C. Lymph node cells (1×10⁶) were serially diluted at 1:3 and cultured overnight in quadruplet wells of ovalbumin-coated nitrocellulose lined 96-well ELISPOT™ plates (Millipore, Bedford, Mass.). Cells were discarded and wells were treated with biotinylated anti-mouse total IgG (Southern Biotech, Birmingham, Ala.) for 1.5 hours at room temperature. Wells were washed and treated with Streptavidin Alkaline phosphatase (Vector Labs) for another 1.5 hours at room temperature. Finally, NBT/BCIP colorimetric substrate for Alkaline phosphatase was added to the wells and the reaction was stopped after visualization of ELISPOTS. The number of ELISPOTS per well was counted using an ELISPOT™ reader. FIG. 28 indicates a synergistic increase in the number of ELISPOTS per 1×10⁶ total lymph node cells in mice treated with the combination of MPL+R837, compared to cells treated with MPL or R837 adjuvants alone, at both day 28 post primary immunization and day 14 post boost immunization. The graph shown in FIG. 29 represents the kinetics of the formation ASCs (ELISPOTS) with the different treatment groups. These results indicate that the combination of MPL+R837 results in synergistic increases in the number of ASCs at day 28 post primary immunization that persists at all time points post boost immunization in the draining lymph nodes. The results shown in FIG. 30 indicate that synergistic increases in the ASCs in the draining lymph nodes upon treatment with MPL+R837 is detectable at 1.5 years post prime and boost immunization.

### Example 19

### Co-Delivery of MPL+R837 Encapsulated in Nanoparticles with Ovalbumin Encapsulated in Nanoparticles Induces Unique Genetic Changes in Class Switched B Cells

To evaluate genetic changes in B cells following administration of TLR ligand- and antigen-containing nanoparticles, microarray analysis was performed. Microarray based genomic analysis of FACS sorted TCRβ⁻CD11b⁻CD19³⁰ IgD⁻IgG⁺ cells demonstrated modulation of genes in TLR ligand-treated mice compared with naïve B cells. Mice treated with the combination of MPL and R837 exhibited the lowest level of altered gene expression on day 7 post primary immunization, but displayed trends of increasingly altered genetic signatures on day 14 compared to single TLR ligand (MPL or R837) treatment.

### Example 20

### Vaccination of a Subject Against Influenza Virus Infection

This example describes the vaccination of a subject against influenza virus infection by administration of PLGA-encapsulated influenza virus antigen and PLGA-encapsulated TLR ligands. To elicit a protective immune response against future exposure to influenza virus, a subject is co-administered PLGA nanoparticles containing the avian influenza protein H5HA, and PLGA nanoparticles containing the TLR4 ligand MPL and the TLR7/TLR8 ligand R837. The dose of H5HA antigen is approximately 10 μg, while the doses of MPL and R837 are approximately 36 μg and 60 μg, respectively. The nanoparticles are administered to the subject intravenously in a pharmaceutically acceptable carrier. A booster dose is administered to the subject approximately one month following the first dose. Subsequent booster doses can be administered as needed to maintain protective immunity over time (indicated, for example, by the presence of high affinity/high avidity antibodies specific for H5HA), such as once a year, once every 5 years or once every 10 years.

### Example 21

### Treatment of a Subject Diagnosed with Prostate Cancer

This example describes the treatment of a subject diagnosed with prostate cancer with nanoparticles containing a prostate cancer-specific antigen and nanoparticles containing a combination of TLR ligands. Following a prostatectomy, a subject with prostate cancer is administered a composition comprising nanoparticles containing prostate-specific antigen (PSA) and nanoparticles containing the TLR4 ligand MPL and the TLR7/TLR8 ligand R837. Administration of antigen-containing and TLR ligand-containing nanoparticles stimulates an immune response in the subject against PSA to prevent recurrence or spread of the prostate cancer. The dose of PSA is approximately 10 μg, while the doses of MPL and R837 are approximately 36 μg and 60 μg, respectively. The nanoparticles are administered to the subject intravenously in a pharmaceutically acceptable carrier. Booster doses are administered to the subject as needed to maintain an effective immune response (indicated by, for example, the presence of PSA-specific CD8⁺ T cells), such as once a month, once every six months, once a year or once every two years.

This disclosure provides a method of enhancing an immune response to an antigen comprising co-delivery of antigen-containing and TLR ligand-containing nanoparticles. The disclosure further provides compositions for eliciting an immune response comprising antigen-containing and TLR ligand-containing nanoparticles. It will be apparent that the precise details of the methods described may be varied or modified without departing from the spirit of the described disclosure. We claim all such modifications and variations that fall within the scope and spirit of the claims below.

1. A composition for stimulating an immune response to an antigen, comprising the antigen, a toll-like receptor (TLR) 4 ligand, and a TLR7/TLR8 ligand, wherein the antigen, TLR4 ligand and TLR7/TLR8 ligand are encapsulated by nanoparticles.

2. The composition of claim 1, wherein the TLR4 ligand is encapsulated in the same nanoparticles as the TLR7/TLR8 ligand.

3. The composition of claim 1, wherein the antigen is encapsulated in different nanoparticles as the TLR ligands.

4. The composition of claim 1, further comprising a pharmaceutically acceptable carrier.

5. The composition of claim 1, wherein the nanoparticles comprise polymeric nanoparticles.

6. The composition of claim 5, wherein the polymeric nanoparticles comprise poly(lactic acid) nanoparticles, poly(glycolic acid) nanoparticles, or both.

7. The composition of claim 5, wherein the polymeric nanoparticles comprise poly(D,L-lactic-co-glycolic acid) (PLGA) nanoparticles.

8. The composition of claim 1, wherein the TLR4 ligand is MPL.

9. The composition of claim 1, wherein the TLR7/TLR8 ligand is R837 or R848.

10. The composition of claim 1, wherein the antigen is a cancer antigen.

11. The composition of claim 10, wherein the cancer is selected from melanoma, breast cancer, prostate cancer and pancreatic cancer.

12. The composition of claim 1, wherein the antigen is an antigen from a pathogen.

13. The composition of claim 12, wherein the antigen is selected from anthrax protective antigen (PA), avian influenza hemagglutinin (H5HA), and H1N1 swine influenza.

14. (canceled)

15. A method of stimulating an immune response to an antigen in a subject, comprising administering to the subject a therapeutically effective amount of the composition of claim 1, thereby stimulating the immune response.

16. The method of claim 15, wherein stimulating an immune response is indicated by an increase in the production of pro-inflammatory cytokines; an increase in the number of CD8⁺ T effector cells; an increase in the number of CD8⁺ T memory cells; an increase in the number of CD4⁺ T effector or memory cells; an increase in titer of antigen-specific antibodies; an increase in antigen-specific antibody affinity; an increase in titer of neutralizing antibodies; an increase in the proliferation of naïve B cells; an increase in persistence of antigen-specific B cells; an increase in the number of germinal centers; or an increase in the number of antibody secreting cells, relative to the absence of the composition, or a combination of two or more thereof.

17. The method of claim 16, further comprising detecting one or more indicators of an immune response in a sample obtained from the subject.

18-21. (canceled)

22. The method of claim 15, wherein the subject has cancer.

23. The method of claim 22, wherein the cancer is selected from melanoma, breast cancer, prostate cancer and pancreatic cancer.

24. (canceled)

25. The method of claim 15, wherein the subject is infected with a pathogen.

26. The method of claim 25, wherein the pathogen is Bacillus anthracis or influenza virus.

27-30. (canceled)
